SEQUENCE LISTING (16 sequences)

SEQ ID NO: 1 - DNA, 23 bp, Rattus Norvegicus
atggtgtcctccaccagcatccc

SEQ ID NO: 2 - DNA, 24 bp, Rattus Norvegicus
ttaagaagaagaattgacgtttcc

SEQ ID NO: 3 - DNA, 20 bp, Rattus Norvegicus
cggcatagcctacaaggtca

SEQ ID NO: 4 - DNA, 20 bp, Rattus Norvegicus
gatcactacggtccgcagaa

SEQ ID NO: 5 - DNA, 22 bp, Rattus Norvegicus
atgggcggtttatactcagagt

SEQ ID NO: 6 - DNA, 21 bp, Rattus Norvegicus
tcagaccactgtgttgccctc

SEQ ID NO: 7 - DNA, 20 bp, Rattus Norvegicus
atctgtgcgctctatgcaag

SEQ ID NO: 8 - DNA, 20 bp, Rattus Norvegicus
tctcggttggtgaaggtgta

SEQ ID NO: 9 - DNA, 20 bp, Mus Musculus
tgccgctctactacctgttc

SEQ ID NO: 10 - DNA, 20 bp, Mus Musculus
aatctcagccgtgtctcctc

SEQ ID NO: 11 - DNA, 23 bp, Rattus Norvegicus
taaagggcatcctgagctacact

SEQ ID NO: 12 - DNA, 23 bp, Rattus Norvegicus
ttactccttggaggccatgtagg

SEQ ID NO: 13 - DNA, 28 bp, Homo Sapiens
ggggtttaaacatggcaactgccctccc

SEQ ID NO: 14 - DNA, 28 bp, Homo Sapiens
ggggtttaaactcagttgcagaagatcc

SEQ ID NO: 15 - DNA, 30 bp, Rattus Norvegicus
ggggtttaaacatgggcggtttatactcag

SEQ ID NO: 16 - DNA, 30 bp, Rattus Norvegicus
ggggtttaaactcagaccactgtgttgccc

(19) United States
(12) Patent Application Publication
(10) Pub. No.: US 2001/0041688 A1
(43) Pub. Date: Nov. 15, 2001

(54) Methods and compositions for the regulation of vasoconstriction

(76) Inventors: Christian Waeber, Boston, MA (US); Michael A. Moskowitz, Belmont, MA (US); Shin-Ichi Yoshimura, Zurich (CH); Salvatore Salomone, Somerville, MA (US)

Correspondence Address: Edward R. Gates, c/o Wolf, Greenfield & Sacks, P.C., Federal Reserve Plaza, 600 Atlantic Avenue, Boston, MA, 02210-2211, US

(21) Appl. No.: 09/804,987

(22) Filed: Mar. 13, 2001

Related U.S. Application Data
(60) Provisional application No. 60/188,859, filed on Mar. 13, 2000.

(51) Int. Cl. A61K 38/19; A61K 31/685

(52) U.S. Cl. 514/78; 424/85.1

(57) ABSTRACT

Methods and compositions for the treatment of conditions which would benefit from vasoconstriction or the inhibition of vasoconstriction via modulation of sphingosine kinase and sphingosine-1-phosphate phosphatase activity and EDG receptor signaling are provided. These conditions include migraine, stroke, subarachnoid hemorrhage and vasospasm. Also provided are screening methods for modulators of sphingosine kinase and sphingosine-1-phosphate phosphatase activity and EDG receptor signaling which are capable of regulating vasoconstriction.

### RELATED APPLICATIONS

This application claims priority under Title 35 §119(e) of the U. S. Provisional Application No. 60/188,859, filed Mar. 13, 2000, and entitled “Methods and Compositions for the Regulation of Vasoconstriction”, the entire contents of which are incorporated herein by reference.

### FIELD OF THE INVENTION

The invention relates to methods and compositions for modulating vasoconstriction for the treatment of vascular occlusive disorders including migraine headaches, stroke, subarachnoid hemorrhage and vasospasm.

### BACKGROUND OF THE INVENTION

Migraine headache (“migraine”) is a common disorder, believed to afflict 20 to 30 percent of the United States population. Almost 80% of migraine sufferers have a family history of such attacks. Migraine headaches are complex conditions and many mechanisms of action have been proposed for their treatment. Although a variety of drug treatments have been developed, none of these drugs has been completely successful in alleviating the symptoms of migraine in the absence of side effects, particularly after long term use. In addition, some of the medications currently available are potentially addictive, making them less desirable for use in children. A real need exists to develop a class of drugs which are effective in treating migraine but do not cause significant side effects and are not addictive.

Stroke and subarachnoid hemorrhage affect 400,000 people annually in the United States alone. Stroke generally refers to a collection of brain disorders having a common underlying cause of an interrupted blood supply to the brain. Stroke alone affects roughly 1 out of every 250 people, and in developed countries it is the third leading cause of death. There is no known cure for stroke. And despite a variety of medications currently available in the treatment of stroke, most are targeted to the treatment of patients after a stroke has occurred or in the prevention of recurrent stroke episodes.

Subarachnoid hemorrhage is a disorder which involves bleeding beneath the membrane covering the brain (i.e., the arachnoid). It occurs in roughly 1 in 10,000 people and is the underlying cause of approximately 5-10% of strokes. Subarachnoid hemorrhage in turn can lead to cerebral vasospasms (i.e., constriction of a blood vessel) for which there are no single effective drugs. One of the most common causes of subarachnoid hemorrhage is traumatic brain injury. Traumatic brain injury is a major cause of disability and is the leading source of brain damage in previously healthy adults in the United States. Motor vehicle accidents account for nearly 50% of all traumatic brain injuries. The second leading cause of traumatic head injury in the United States is firearm related injuries. Falls account for a large proportion of non-fatal traumatic head injuries. Ten million people in the United States suffer head injuries yearly, of which 500,000 require hospitalization.

A common feature of the afore-mentioned disorders is a perturbation of normal blood circulation. Migraine headaches are preceded by a constriction of the cerebrovasculature followed by a vasodilation which coincides with the severe headache experienced by migraine sufferers. Stroke, subarachnoid hemorrhage and vasospasm are disorders which are associated with either a vasoconstriction or a lack of blood supply to a part of the body, particularly the brain.

Therapeutic compounds for the prevention or treatment of these disorders which are safe, non-addictive, and effective and to which the body is not refractory in the long-term are not currently available and would be useful.

### SUMMARY OF THE INVENTION

The invention relates in a broad sense to the control of blood flow in tissues by modulating vasoconstriction and/or vasodilation. More specifically, the invention involves methods and compositions for increasing or, depending on the subject and the disorder to be treated, decreasing blood flow into and within particular tissues such as the brain. Accordingly, the invention relates to vasoconstrictive (or vasodilative, as the case may be) control in particular tissues including, for example, the brain.

The invention is premised, in part, on the finding that sphingosine-1-phosphate, a ligand for some members of the EDG (Endothelial Differentiation Gene) receptor family, is able to cause the selective constriction of cerebral arteries such as the basilar artery and the middle cerebral artery, but not normal peripheral arteries such as the femoral, carotid or coronary arteries. EDG-1, EDG-3, EDG-5, and EDG-8 receptors are all expressed in cerebral arteries, including the basilar artery, and the middle cerebral artery. It was further discovered, in accordance with the invention, that EDG receptors, particularly EDG-3 receptor, are involved in sphingosine-1-phosphate induced vasoconstriction of cerebral arteries, as demonstrated by the ability of anti-sense molecules specific for EDG receptors, particularly EDG-3 receptor, to block the effects of sphingosine-1-phosphate. Interestingly, EDG-3, as well as other EDG receptors, are expressed in other arteries that are non-responsive to sphingosine-1-phosphate, including the coronary, carotid and femoral arteries. This indicates that factors in addition to EDG receptors are involved in the selective vasoconstriction of cerebral arteries.

In attempting to understand the underlying mechanism for the selective vasoconstriction, it has been further discovered that cerebral arteries express lower levels of sphingosine-1-phosphate phosphatase than do the sphingosine-1-phosphate non-responsive arteries. Sphingosine-1-phosphate phosphatase is an enzyme that dephosphorylates sphingosine-1-phosphate, leaving sphingosine as a product. The effect of this enzyme is to reduce the level of sphingosine-1-phosphate, thereby blocking its vasoconstrictive effects, and potentially increasing the level of sphingosine, thereby enhancing its vasodilative effects. Arteries which express lower levels of sphingosine-1-phosphate phosphatase are less able to counteract the vasoconstrictive effects of sphingosine-1-phosphate, as compared to arteries that express higher levels of sphingosine-1-phosphate phosphatase. Thus, when exposed to sphingosine-1-phosphate the former category of arteries constrict, while the latter category do not.

In accordance with these discoveries, the invention provides, inter alia, methods for regulating cerebral vasoconstriction and vasodilation by targeting enzymes and receptors involved in the sphingosine-1-phosphate pathway, including sphingosine, sphingosine-1-phosphate, sphingosine kinase, sphingosine-1-phosphate phosphatase, and EDG receptors. Blood flow through cerebral arteries, including the basilar and the middle cerebral arteries, is particularly targeted by the methods of the invention.

In one aspect, the invention provides a method for treating a subject having, or at risk of having, a disorder which can be treated by increased vasoconstriction or inhibition of vasodilation. The method may comprise administering to a subject in need of such treatment an agent that up-regulates EDG receptor (particularly, EDG-3 receptor) signaling in an amount effective to treat the disorder.

In a related aspect, the invention provides a method for decreasing arterial blood flow in a subject who would benefit from decreased arterial blood flow. The method may comprise administering to a subject in need of such treatment an agent that up-regulates EDG receptor (particularly, EDG-3 receptor) signaling in an amount effective to decrease arterial blood flow.

In another related aspect, the invention provides a method for inducing vasoconstriction in a subject who would benefit from induced vasoconstriction. The method may comprise administering to a subject in need of such treatment an agent that up-regulates EDG receptor (particularly EDG-3 receptor) signaling in an amount effective to induce vasoconstriction.

Agents that up-regulate EDG receptor signaling embrace a number of agents including those that bind and directly affect EDG receptor including EDG receptor agonists (e.g., naturally occurring ligands such as sphingosine-1-phosphate), those that affect downstream signals of EDG receptor signaling, and those that increase the level of EDG receptor agonists, for example, by stimulating the production of such agonists. An example of this last category of agents is sphingosine kinase activators which are agents that stimulate (i.e., up-regulate) the activity of sphingosine kinase, the enzyme that produces sphingosine-1-phosphate. Thus, in one embodiment, the agent is a sphingosine kinase activator. In an important embodiment, the sphingosine kinase activator is TNF-α or EGF.

In another embodiment, the agent is an EDG receptor agonist. An EDG receptor agonist is a compound that binds to and activates an EDG receptor. As an example, an EDG-3 receptor agonist is a compound that binds to and activates an EDG-3 receptor. In important embodiments, the EDG receptor agonist is specific for a single EDG receptor and neither binds to nor activates other EDG receptors. The invention embraces EDG-1, EDG-3, EDG-5, and EDG-8 receptor agonists. In a preferred embodiment, the EDG receptor agonist is an EDG-3 receptor agonist. In important embodiments, the EDG receptor agonist is selected from the group consisting of sphingosine-1-phosphate, dihydro-sphingosine-1-phosphate, a sphingosine-1-phosphate analog, psychosine, sphingosylphosphorylcholine and lysophosphatidic acid. In certain embodiments, the EDG receptor agonist is sphingosine-1-phosphate or dihydro-sphingosine-1-phosphate.

In yet another embodiment, the agent is a sphingosine-1-phosphate phosphatase inhibitor. A sphingosine-1-phosphate phosphatase inhibitor is a compound that reduces activity of sphingosine-1-phosphate phosphatase. This may be accomplished by reducing the level of sphingosine-1-phosphate phosphatase and/or by inhibiting its activity directly. An example of a sphingosine-1-phosphate phosphatase inhibitor is an agent that binds to sphingosine-1-phosphate phosphatase and thereby inhibits its enzymatic activity (i.e., a sphingosine-1-phosphate phosphatase antagonist).

Arterial blood flow refers to the blood flow in and through an artery. In important embodiments, the arterial blood flow is cerebral artery blood flow. Cerebral artery blood flow refers to the blood flow in and through a cerebral artery. A cerebral artery may be selected from the group consisting of the basilar artery, the internal and external carotid arteries, the anterior cerebral artery, the middle cerebral artery, the posterior cerebral artery, the vertebral artery, the posterior inferior cerebellar artery and the middle meningeal artery. In an important embodiment, the cerebral artery is a basilar artery or a middle cerebral artery. In another embodiment, vasoconstriction refers to arterial vasoconstriction (i.e., the vasoconstriction of an artery). In a preferred embodiment, the arterial vasoconstriction is cerebral artery vasoconstriction (e.g., basilar artery vasoconstriction and middle cerebral artery vasoconstriction).

In one further embodiment, the subject is having, or is at risk of having, a migraine headache.

In one aspect, the invention further provides a method for treating a subject having, or at risk of having, a disorder which can be treated by increased vasodilation or inhibition of vasoconstriction. The method may involve administering to a subject in need of such treatment an agent that down-regulates EDG receptor, particularly EDG-3 receptor, signaling in an amount effective to treat the disorder.

In a related aspect, the invention provides a method for increasing arterial blood flow in a subject who would benefit from increased arterial blood flow. The method may involve administering to a subject in need of such treatment an agent that down-regulates EDG receptor, particularly EDG-3 receptor, signaling in an amount effective to increase arterial blood flow.

In another related aspect, the invention further provides a method for inhibiting vasoconstriction in a subject who would benefit from inhibited vasoconstriction. The method may comprise administering to a subject in need of such treatment an agent that down-regulates EDG receptor, particularly EDG-3 receptor, signaling in an amount effective to inhibit vasoconstriction.

An agent that down-regulates EDG receptor signaling embraces agents that interfere with EDG receptor signaling (i.e., antagonists) either by binding EDG receptor directly or by negatively affecting downstream signals of EDG receptor signaling (i.e., functional antagonists), as well as agents that decrease the level of EDG receptor agonists, for example, by inhibiting the production of such agonists. An example of this last category of agents is sphingosine kinase inhibitors which are agents that interfere or down-regulate the activity of sphingosine kinase. Thus, in one important embodiment, the agent is sphingosine kinase inhibitor. In another embodiment, the sphingosine kinase inhibitor is selected from the group consisting of N,N-dimethylsphingosine, D,L-threo-dihydrosphingosine, high density lipoprotein, and 3-fluoro-sphingosine analogues. Another example of an agent that down-regulates EDG receptor signaling is an anti-sense molecule to an EDG receptor, particularly an anti-sense molecule to an EDG-3 receptor. As shown in the Examples, this latter agent is effective in inhibiting sphingosine-1-phosphate induced vasoconstriction.

In another important embodiment, the agent is an EDG receptor antagonist. In one embodiment, the EDG receptor antagonist is selected from the group consisting of an EDG-1 receptor antagonist, an EDG-3 receptor antagonist, an EDG-5 receptor antagonist and an EDG-8 receptor antagonist. In a preferred embodiment, the EDG receptor antagonist is an EDG-3 receptor antagonist. In yet another preferred embodiment, the EDG-3 receptor antagonist is a functional antagonist selected from the group consisting of sphingosine or suramin.

In yet another embodiment, the agent is a sphingosine-1-phosphate phosphatase activator. A sphingosine-1-phosphate phosphatase activator is a compound that increases the activity of sphingosine-1-phosphate phosphatase. An example of a sphingosine-1-phosphate phosphatase activator is an agent that binds to sphingosine-1-phosphate phosphatase directly and thereby activates (i.e., increases) its activity. Such an agent is referred to as a sphingosine-1-phosphate phosphatase agonist. Other types of sphingosine-1-phosphate phosphatase activators are agents that increase the level of sphingosine-1-phosphate phosphatase (and thereby indirectly increase the level of sphingosine-1-phosphate phosphatase activity).

In an important embodiment, the arterial blood flow is cerebral artery blood flow. Cerebral artery blood flow may be basilar artery blood flow (i.e., the blood flow into and through a basilar artery) or middle cerebral artery blood flow (i.e., the blood flow into and through a middle cerebral artery), but is not so limited.

In certain embodiment, the subject is one having a disorder that can be treated by increased cerebral vasodilation or inhibition of cerebral vasoconstriction. Cerebral vasodilation or vasoconstriction may occur in a cerebral artery such as but not limited to a basilar artery, a middle cerebral artery, an internal carotid artery, a posterior cerebral artery, and a middle meningeal artery. In a further embodiment, the subject is having, or is at risk of having, a stroke, a subarachnoid hemorrhage or a vasospasm. In some important embodiments, the vasospasm is a cerebral vasospasm.

In certain embodiments, the methods further comprise co-administering a second agent to the subject with a condition treatable by the second agent in an amount effective to treat the condition, whereby the delivery of the second agent to a tissue of the subject is enhanced as a result of the increased blood flow. The second agent may be selected from the group consisting of analeptic, analgesic, anesthetic, adrenergic agent, anti-adrenergic agent, amino acids, antagonists, antidote, anti-anxiety agent, anti-cholinergic, anti-convulsant, anti-depressant, anti-emetic, anti-epileptic, anti-hypertensive, anti-fibrinolytic, anti-hyperlipidemia, anti-nauseant, anti-neoplastic (brain cancer), anti-obsessional agent, anti-obesity, anti-parkinsonian, anti-psychotic, appetite suppressant, blood glucose regulator, cognition adjuvant, cognition enhancer, dopaminergic agent, emetic, free oxygen radical scavenger, glucocorticoid, hypocholesterolemic, hypolipidemic, histamine H2 receptor antagonists, immunosuppressant, memory adjuvant, mental performance enhancer, mood regulator, mydriatic, neuromuscular blocking agent, neuroprotective, NMDA antagonist, post-stroke and post-head trauma treatment, psychotropic, sedative, sedative-hypnotic, serotonin inhibitor, tranquilizer, calcium channel blockers, free radical scavengers (e.g., anti-oxidants), GABA agonists, glutamate antagonists, AMPA antagonists, kainate antagonists, competitive and non-competitive NMDA antagonists, growth factors, opioid antagonists, phosphatidylcholine precursors, serotonin agonists, sodium- and calcium-channel blockers, and potassium channel openers.

According to other aspects of the invention, methods are provided for identifying agents that regulate vasoconstriction. In one aspect, the method comprises selecting an agent that binds to sphingosine kinase, and determining whether the agent that binds to sphingosine kinase modulates vasoconstriction. In another aspect, the method comprises selecting an agent that binds to an EDG receptor (preferably an EDG-3 receptor), and determining if the agent that binds to the EDG receptor modulates vasoconstriction. In yet another aspect, the method comprises selecting an agent that binds to sphingosine-1-phosphate phosphatase, and determining if the agent that binds to sphingosine-1-phosphate phosphatase modulates vasoconstriction. Modulation of vasoconstriction can be determined by recording of isometric tension in isolated blood vessels or by recording of intraluminal pressure in perfused isolated vessels. A change in vasoconstriction in the presence of the agent is indicative of an agent that regulates vasoconstriction. In one embodiment, the agent is a lipid. In another embodiment, the agent is a library member. In yet another embodiment, the library is a combinatorial chemical library.

In another aspect, the invention provides a pharmaceutical preparation comprising an agent that up-regulates EDG receptor (preferably EDG-3 receptor) signaling in an effective amount to treat a disorder, and a pharmaceutically-acceptable carrier. The disorder is one which can be treated by increased vasoconstriction or inhibition of vasodilation. In some embodiments, the disorder may be further characterized by abnormal vasodilation. In one embodiment, the disorder is one that can be treated by increased cerebral vasoconstriction or inhibition of cerebral vasoconstriction. In an important embodiment, the disorder is a migraine headache.

In another aspect, a pharmaceutical preparation is provided which comprises an agent that down-regulates EDG receptor signaling in an effective amount to treat a disorder, and a pharmaceutically-acceptable carrier. The disorder is one which is treated by increased vasodilation or inhibition of vasoconstriction. In one embodiment, the disorder may be further characterized by abnormal vasoconstriction. In one embodiment, the disorder is one which is treated by increased cerebral vasodilation or inhibition of cerebral vasoconstriction. In an important embodiment, the disorder is selected from the group consisting of stroke, subarachnoid hemorrhage and a vasospasm. In another embodiment, the vasospasm is a cerebral vasospasm. In still another embodiment, the pharmaceutical preparation also comprises a second therapeutic agent selected from the group listed above which is intended to treat a disorder and delivery of which is facilitated by combined administration with an agent that down-regulates EDG receptor signaling.

The invention further intends to embraces kits comprising the pharmaceutical preparations of the invention in a housing and instructions for use.

### Abbreviated Sequence Listing

SEQ ID NO:1 is the nucleotide sequence for a sense primer for rat edg-1.

SEQ ID NO:2 is the nucleotide sequence for an antisense primer for rat edg-1.

SEQ ID NO:3 is the nucleotide sequence for a sense primer for rat edg-3.

SEQ ID NO:4 is the nucleotide sequence for an antisense primer for rat edg-3.

SEQ ID NO:5 is the nucleotide sequence for a sense primer for rat edg-5.

SEQ ID NO:6 is the nucleotide sequence for an antisense primer for rat edg-5.

SEQ ID NO:7 is the nucleotide sequence for a sense primer for rat edg-8.

SEQ ID NO:8 is the nucleotide sequence for an antisense primer for rat edg-8.

SEQ ID NO:9 is the nucleotide sequence for a sense primer for mouse spp1.

SEQ ID NO:10 is the nucleotide sequence for an antisense primer for mouse spp1.

SEQ ID NO:11 is the nucleotide sequence for a sense primer for rat gapdh.

SEQ ID NO:12 is the nucleotide sequence for an antisense primer for rat gapdh.

SEQ ID NO:13 is the nucleotide sequence for a sense primer for human edg-3 (plus PmeI site).

SEQ ID NO:14 is the nucleotide sequence for an antisense primer for human edg-3 (plus PmeI site).

SEQ ID NO:15 is the nucleotide sequence for a sense primer for rat edg-5 (plus PmeI site).

SEQ ID NO:16 is the nucleotide sequence for an antisense primer for rat edg-5 (plus PmeI site).

### BRIEF DESCRIPTION OF THE DRAWINGS

FIG. 1A is a dose response curve showing the effect of sphingosine-1-phosphate (SIP) on contractile response in basilar (open circles), middle cerebral (closed circles), coronary (open squares), carotid (closed squares) and femoral (open triangles) arteries. Points represent mean±s.e.m. from n preparations as reported in Table 1.

FIG. 1B is a dose response curve showing the effect of dihydrosphingosine-1-phosphate (DHS1P) on contractile response in basilar (open circles), middle cerebral (closed circles), coronary (open squares), carotid (closed squares) and femoral (open triangles) arteries. Points represent mean±s.e.m. from n preparations as reported in Table 1.

FIG. 1C is a photograph of an RT-PCR gel showing EDG-1, EDG-3, EDG-5 and EDG-8 receptor mRNA in basilar (BA), carotid (CA), femoral (FA), coronary (Cor) arteries and aorta (Ao). GAPDH represents glyceraldehyde-3-phosphate dehydrogenase. Identical results have been obtained with 3 independent RNA extracts.

FIG. 1D is a photograph of an RT-PCR gel showing S1P phosphatase mRNA in basilar (BA), carotid (CA), femoral (FA), coronary (Cor) arteries and aorta (Ao). PCR products at different cycle number (36, 38, 40) show that the amplification occurs within the exponential phase (i.e., quantitative and non-saturated phase) of the reaction.

FIG. 1E is a bar graph showing the results from 4 independent RNA extracts are (mean±s.e.m.).

FIG. 2 is a dose response curve showing the effects of sphingosine and the sphingosine kinase inhibitor N,N-dimethylsphingosine on sphingosine-1-phosphate-induced vasoconstriction. The contractile response to sphingosine-1-phosphate (S1P) was measured in isolated basilar arteries in the absence (control, open circles) or in the presence of sphingosine (10 μM, closed circles), N,N-dimethyl-sphingosine (DMS, 20 μM, open squares) or sphingosine plus DMS (closed squares). When used, sphingosine and/or DMS were added to the organ bath 30 min prior to S1P. Points represent mean±s.e.m. from 4-6 preparations. The DMS treated group was significantly different from control, and the sphingosine plus DMS group was significantly different from both control and DMS alone (p<0.05, two-way ANOVA followed by Tukey post hoc test).

FIG. 3A is a photograph of an RT-PCR gel showing EDG-3, EDG-5 and glyceraldehyde-3-phosphate dehydrogenase (GAPDH) expression from RNA extracted from adenovirus-untreated (CTR) basilar arteries, and basilar arteries treated with the empty vector (EV), the virus bearing edg-3 antisense (3AS) and the virus bearing edg-5 antisense (5AS).

FIG. 3B is a dose response curve showing the contractile response of isolated basilar arteries to sphingosine-1-phosphate (S1P) after the following treatments: adenovirus-untreated (CTR); empty vector (EV); virus bearing edg-3 antisense (3AS); virus bearing edg-5 antisense (5AS). Points represent mean±s.e.m. from 8 preparations. The response of basilar arteries treated with a virus bearing edg-3 antisense (3AS) was significantly different from all other treatment groups (p<0.05, two-way ANOVA followed by Tukey post hoc test).

FIG. 4A is a dose-response curve showing the in vitro effects of suramin on sphingosine-1-phosphate-induced vasoconstriction. The contractile response to sphingosine-1-phosphate (S1P) was measured in isolated basilar arteries in the absence (control, open squares) or in the presence of 10 μM suramin (closed squares). When used, suramin was added to the organ bath 30 min prior to S1P. Points represent mean±s.e.m. from 4-6 preparations.

FIG. 4B is a time course graph showing the effects of S1P (0.3 mg/kg, open circles), DHS1P (0.3 mg/kg, closed circles) or S1P (0.3 mg/kg) plus suramin (16 mg/kg) (open squares) on relative cerebral blood flow (CBF) compared to vehicle alone (control, closed squares), as measured by laser Doppler flowmetry in anesthetized rats. Mean arterial blood pressure, heart rate and blood gases were not affected by the treatments. Points represent mean±s.e.m. from 5-7 animals. S1P treatment was significantly different from control/vehicle, and DHS1P treatment was significantly different from control and S1P treatments (p<0.05, two-way ANOVA followed by Tukey post hoc test).

### The drawings are not required for enablement of the claimed invention.

### DETAILED DESCRIPTION OF THE INVENTION

The invention provides methods for the treatment of disorders which would benefit from either an increase or an inhibition of vasoconstriction or vasodilation. Some of these disorders may be further characterized by inappropriate, or detrimental, vasoconstriction or vasodilation. In specific aspects, the methods and compositions of the invention aim to regulate cerebral vasoconstriction, vasodilation and resultant blood flow.

The invention results from the surprising finding that exposure of cerebral arteries, such as the basilar artery and the middle cerebral artery, to the sphingolipids, sphingosine-1-phosphate (S1P) and dehydrosphingosine-1-phosphate (DS1P), results in preferential constriction of the arteries. As shown in the Examples, the vasoconstrictive effect of these compounds on other arteries of the body (e.g., peripheral arteries), including the femoral artery, the carotid artery and the coronary artery, was less than that in the basilar artery and the middle cerebral artery. This unexpected finding indicates that S1P and its mechanism of action are candidate targets for controlling cerebral vasoconstriction and the associated cerebral blood flow. Yet another unexpected finding was the observation that sphingosine was able to antagonize the effect of S1P or DS1P in the arteries tested and specifically in cerebral arteries. These findings indicate that both S1P and DS1P can be used to induce vasoconstriction of cerebral arteries including but not limited to the basilar artery and the middle cerebral artery, and that sphingosine can be used as an antagonist of this activity. Consistent with such observations is the idea that pathways which modulate sphingosine and S1P production can also be targeted in order to regulate cerebral blood flow. Sphingosine-1-phosphate is produced either de novo or through the action of sphingosine kinase on ceramide or sphingosine. Sphingosine is produced either de novo or through the action of sphingosine-1-phosphate phosphatase on sphingosine-1-phosphate. Thus, agonists and antagonists of sphingosine kinase can be used to treat conditions which can be treated by increased or decreased vasoconstriction, respectively. Similarly, agonists and antagonists of sphingosine-1-phosphate phosphatase can be used to treat conditions that can be treated by decreased or increased vasoconstriction, respectively.

To further elucidate the basis for S1P and DS1P specificity for cerebral arteries, the expression profile of cerebral artery cells was analyzed. It was found that several EDG receptor family members are expressed in cerebral arteries, such as the basilar and middle cerebral arteries. EDG receptors were regarded as orphan receptors prior to the identification of their endogenous ligands. At present, a number of endogenously occurring ligands have been identified for each receptor type, including sphingosine-1-phosphate for EDG-1, EDG-3, EDG-5, EDG-6, and EDG-8, and lysophosphatidic acid (LPA) for EDG-2 and EDG-4. EDG-1, EDG-3 and EDG-5 are able to bind to LPA but only when LPA is present at very high concentrations, suggesting that EDG-1, EDG-3 and EDG-5 have a lower affinity for LPA as compared to sphingosine-1-phosphate.

EDG receptors are G coupled proteins made up of seven transmembrane, and thus hydrophobic, antiparallel α helices. These transmembrane segments impart the structural and functional features of the receptor, including co-operatively forming the ligand binding cleft of the receptor. When bound by their respective ligands, EDG receptors interact with intracellular G-proteins. Each EDG receptor interacts specifically with one or more G proteins. EDG receptor signaling ultimately results in production and/or release of second messengers such as cyclic AMP (cAMP), IP₃ and Ca²⁺, and thus can be measured by the production and/or release of these and other second messengers.

It was discovered that some EDG receptors, including EDG-1, EDG-3, EDG-5 and EDG-8 receptors, are expressed in cerebral arteries. Cerebral arteries include but are not limited to the internal carotid artery, the middle cerebral artery, the posterior cerebral artery, the basilar artery and the middle meningeal artery. Anti-sense experiments aimed at determining which EDG receptors are involved in sphingosine-1-phosphate induced vasoconstriction were performed, as described in the Examples. It was found that anti-sense molecules specific for EDG-3 receptor were able to inhibit sphingosine-1-phosphate induced vasoconstriction, indicating that at least EDG-3 receptor was involved in the biological response. These novel observations indicate that targeting of EDG receptors, and particularly EDG-3 receptor, can also be used for therapeutic purposes in regulating cerebral vasoconstriction and vasodilation and accompanying cerebral blood flow.

The expression profiles of cerebral arteries also indicated that sphingosine-1-phosphate phosphatase was expressed in cerebral arteries at a much lower level than in peripheral, such as femoral, carotid and coronary arteries, which also expressed EDG-3. Sphingosine-1-phosphate phosphatase is an enzyme that dephosphorylates sphingosine-1-phosphate, and in doing so produces sphingosine. Although not intending to be bound by any particular theory, it is thought that cerebral arteries are responsive to sphingosine-1-phosphate, in part, because they possess low levels of sphingosine-1-phosphate phosphatase and thus are not able to convert this molecule to sphingosine. Peripheral arteries which express higher levels of this enzyme are able to convert sphingosine-1-phosphate to sphingosine and thereby circumvent the vasoconstrictive effects of the molecule. Accordingly, the novel observations indicate that targeting of sphingosine-1-phosphate phosphatase can also be used for prophylactic and therapeutic purposes in regulating cerebral vasoconstriction and vasodilation and accompanying cerebral blood flow. Additionally, sphingosine-1-phosphate phosphatase can also be targeted in peripheral arteries if the desired purpose is to vasoconstrict such arteries.

The methods of the invention are useful in both the therapeutic and the prophylactic treatment of particular conditions. As used herein, a therapeutic treatment refers to the treatment of subjects having a particular condition. Prophylactic treatment refers to the treatment of subjects at risk of having a particular condition, which may include a patient with a history of having such a condition, but not presently experiencing the symptoms of the condition. The agents of the invention can be administered to subjects in either an acute or a chronic manner.

In its broadest sense, the terms “treatment” or “to treat” refer to both therapeutic and prophylactic treatments. If the subject in need of treatment is experiencing a condition (i.e., has or is having a particular condition), then “treating the condition” refers to ameliorating, reducing or eliminating one or more symptoms arising from the condition. In some preferred embodiments, treating the condition refers to ameliorating, reducing or eliminating a specific symptom or a specific subset of symptoms associated with the disorder. If the subject in need of treatment is one who is at risk of having a condition, then treating the subject refers to reducing the risk of the subject having the condition.

As used herein, a subject includes humans, non human primates, dogs, cats, sheep, goats, cows, pigs, horses and rodents. In preferred embodiments, the subject is human.

According to one aspect of the invention, compounds useful in promoting or maintaining vasoconstriction are agents which up-regulate EDG receptor signaling. Agents which up-regulate EDG receptor signaling include EDG receptor activators, sphingosine kinase activators, and sphingosine-1-phosphate phosphatase inhibitors.

Sphingosine kinase is a protein which produces sphingosine-1-phosphate by the phosphorylation of sphingosine. Sphingosine kinase activators are compounds which up-regulate the activity, particularly the kinase activity, of sphingosine kinase. Kinase activity refers to the phosphorylation of substrates, and in the present case, kinase activity of sphingosine kinase results in the synthesis of sphingosine-1-phosphate. Agents that activate sphingosine kinase can function at a number of levels and in a number of different pathways including transcription and translation of sphingosine kinase genes and transcripts and post-translational modifications of sphingosine kinase. Other sphingosine kinase activators are sphingosine kinase agonists. Sphingosine kinase agonists bind sphingosine kinase and thereby enhance its kinase activity, ultimately upregulating the production of sphingosine-1-phosphate. Assays for transcriptional and/or translational activating factors have been described in the literature with respect to other genes. It is well within the skill of the ordinary artisan to adapt such techniques to the identification of activating factors specific for sphingosine kinase. Methods for measuring sphingosine kinase activity as well as methods for identifying sphingosine kinase agonists and antagonists are disclosed in PCT Patent Application No. PCT/AU98/00730 (WO 99/12533), the contents of which are incorporated herein by reference in their entirety. Examples of sphingosine kinase activators are tumor necrosis factor—alpha (i.e., TNF-α), epidermal growth factor (i.e., EGF), and platelet derived growth factor (i.e., PDGF).

One of the ultimate aims of activating sphingosine kinase is to increase the amount of sphingosine-1-phosphate in, for example, cerebral artery cells. Sphingosine-1-phosphate is a natural activating ligand for a subset of EDG receptors, including EDG-1, EDG-3, and EDG-5. As shown in the Examples, exposure of cerebral arteries to sphingosine-1-phosphate results in their constriction, a phenomenon which would be useful in the treatment of migraine headaches.

Thus, yet other agents useful in the treatment of disorders or conditions which would benefit from vasoconstriction, particularly cerebral vasoconstriction, are those that activate EDG receptors (i.e., EDG receptor activators). Agents useful in the treatment of disorders which would benefit, and thus which can be treated, by increased vasoconstriction or the inhibition of vasodilation include, but are not limited to, agents which up-regulate the cell surface expression of EDG receptors, or which enhance the interactions of EDG receptors with other proteins, particularly when such interactions are involved in EDG signaling. In preferred embodiments, the agents preferentially affect EDG-1 receptors, EDG-3 receptors, EDG-5 receptors and EDG-8 receptors. Agents which specifically impact upon EDG-3 cell surface expression, protein interactions, and/or signaling are even more preferred, according to some embodiments.

A preferred EDG receptor activator is an EDG receptor agonist. As used herein, an EDG receptor agonist is an agent which binds to EDG receptor and thereby activates, for example, an endogenous enzymatic activity such as a kinase activity or a signaling pathway leading from the receptor. EDG receptor agonists, as used herein, embrace naturally occurring EDG receptor ligands. In a preferred embodiment, the EDG receptor agonist is one which binds and thereby activates EDG-1 receptor, EDG-3 receptor, EDG-5 receptor or EDG-8 receptor. In an even more preferred embodiment, the agent is an EDG-3 receptor agonist. The EDG-3 receptor has been cloned and its nucleotide sequence is known (e.g., Genbank Accession Number AF184914). The EDG-1 and EDG-5 receptors have similarly been cloned and their nucleotide sequences are publicly available as GenBank Accession Numbers RNU10303 and AB016931. Agonists for some EDG receptors are known in the art. EDG-3 receptor agonists include but are not limited to native ligands such as sphingosine-1-phosphate, sphingosylphosphorylcholine, and psychosine. EDG-1 receptor agonists include but are not limited to native ligands such as sphingosine-1-phosphate and sphingosylphosphorylcholine. EDG-5 receptor agonists include but are not limited to native ligands such as sphingosine-1-phosphate. At high concentrations, lysophosphatidic acid is also a ligand for some of the EDG receptors.

Another important category of agents useful in the afore-mentioned methods are sphingosine-1-phosphate phosphatase inhibitors. Sphingosine-1-phosphate phosphatase inhibitors are agents that reduce or inhibit completely the activity of sphingosine-1-phosphate phosphatase. Such inhibitors may do so by affecting the transcriptional, translational and/or post-translational mechanisms involved in sphingosine-1-phosphate phosphatase expression. Additionally, suitable inhibitors may also affect the enzyme activity directly without affecting the expression level of the enzyme. This latter class of inhibitors includes agents that bind to sphingosine-1-phosphate phosphatase and inhibit its activity and are referred to as sphingosine-1-phosphate phosphatase antagonists.

The invention, in one aspect, provides methods for treating disorders or conditions which would be benefit from, and thus which can be treated by, increased vasoconstriction or inhibition of vasodilation. Some of these disorders may be further characterized by detrimental vasodilation. An example of a condition which can be treated by increased vasoconstriction or inhibition of vasodilation is a migraine headache. As used herein, the terms migraine headache, migraine and migraine attacks are used interchangeably.

A migraine headache is the most common type of vascular headache. It is associated with changes in the diameter of blood arteries leading to and from the brain, as well as those within the brain. It encompasses both classic and common migraine headaches and involves the abnormal sensitivity of blood vessels (i.e., arteries) in the brain to various stimuli. This abnormal sensitivity ultimately results in rapid changes in artery size (i.e., a spasm or vasoconstriction). Following this initial constriction, other arteries in the brain and scalp dilate, creating a perceived throbbing pain in the head. Migraine tendency is inherited and appears to involve serotonin, a chemical in the brain involved in the transmission of nerve impulses.

The invention in one aspect provides a method to treat a subject having (i.e., experiencing) a migraine headache. Migraines are most commonly associated with symptoms such as an intense throbbing headache (often on one side of the head only, and therefore referred to as unilateral), nausea and vomiting, increased sensitivity to light, sounds and smell. Migraine headaches are also associated with visual disturbances which are collectively called an aura. As used herein, migraine-associated symptoms include the preceding list of symptoms. A subject having a migraine is defined herein as a person experiencing two or more migraine-associated symptoms, wherein preferably, one of the symptoms is a severe or throbbing headache. Migraine headaches usually last anywhere from a couple of hours to a couple of days. The vast majority of migraine sufferers have a personal and/or family history of migraine headaches. A personal history of migraine headaches means that the subject has had a migraine headache before. A family history of migraine headaches means that at least one member of the subject's family, including a parent, sibling or grandparent, has experienced a migraine headache. Thus a subject having a migraine may also be a subject who is experiencing at least a severe or throbbing headache, and who may optionally have a personal or family history of migraine headache.

In one aspect, the invention provides a method to treat a subject having a migraine headache. As used herein, a subject having a migraine is treated by ameliorating, reducing, or completely eliminating one or more migraine-associated symptoms. Preferably, a subject having a migraine is treated using the agents of the invention to ameliorate, reduce, or completely eliminate at least the throbbing head pain associated with a migraine headache.

In another aspect, the invention provides a method to treat a subject at risk of having a migraine headache. Most people have the capacity to experience a migraine headache. However, in some subjects, perhaps as a result of a genetic predisposition, the threshold for triggering a migraine is lower and such headaches occur more easily and more frequently. Twice as many women as men suffer from migraine headaches, possibly due to the involvement of hormonal factors. Subjects at risk of having a migraine include those that have a personal and/or a family history of migraine, as defined above.

Since many factors have been identified which trigger migraines, it is sometimes possible for a subject to predict whether a certain activity or environment is likely to induce a migraine headache. As a result, a subject at risk of having a migraine headache also embraces a subject who is engaged in an activity, or a subject who is present in an environment, which is likely to trigger a migraine. Subjects at risk of having a migraine especially include subjects with a personal and/or family history of migraine who engage in activities, or are present in environments which trigger migraines. Factors known to trigger migraines include diet or change in eating patterns, particularly fasting or sporadic meals; intake of tyramine which is present in red wines, most alcoholic beverages, aged cheeses, processed meats; intake of food additives such as nitrates, nitrites and monosodium glutamate; intake of alcohol, chocolate, caffeine, or coffee; exposure to sunlight; exercise; physical or mental fatigue; change in sleep patterns (e.g., oversleeping or lack of sleep); tension or stress and in some instances the relief of stress; hormonal changes in menses, the use of oral contraceptives, hormone replacement therapy or menopause; extreme emotions (e.g., grief, anger, etc.); sensory stimuli (e.g., loud noise, bright or flickering lights, strong perfumes, hot stuffy atmosphere, etc.); and changes in climactic conditions (e.g. changes in barometric pressure, changes in altitude, strong winds, extreme heat or cold). It is unusual for any of these factors when experienced individually to induce a migraine. Rather, a combination of these factors must usually be experienced together or in a short period of time in order to trigger a migraine headache. In some instances, however, particularly with persons at an abnormally elevated risk of having a migraine, single factors may be sufficient for triggering the migraine headache. A subject who may reasonably predict that a migraine headache will likely follow as a result of the activity or activities engaged in is, in some embodiments, a preferred subject for prophylactic treatment.

Some migraine sufferers report experiencing an aura roughly five to thirty minutes prior to the onset of pain. An aura may manifest itself in visual, audio or olfactory forms, but is not so limited. Examples of aura manifestations are listed above. Thus the existence of an aura may be used as an indication that a subject is having a migraine (particularly if it is accompanied by another migraine-associated symptom) or that the subject is at risk of having a migraine headache.

The agents useful in the proceeding aspects of the invention are those which up-regulate EDG receptor signaling. Examples of agents which up-regulate EDG receptor signaling include sphingosine kinase activators, sphingosine-1-phosphate phosphatase inhibitors and EDG receptor agonists. To be useful in the methods of the invention, such agents are administered to subjects in effective amounts. In some aspects of the invention, these agents are administered in effective amounts to treat the disorder (e.g., the migraine headache). If the subject is having a migraine headache, and the treatment is acute, then an effective amount is an amount which treats the migraine headache. To treat a migraine, as used herein, means to ameliorate, reduce or eliminate altogether one or more symptoms associated with a migraine headache. Symptoms which are associated with migraines (i.e., migraine-associated symptoms) are listed above. Preferably, the effective amount is the amount which ameliorates, reduces or eliminates the throbbing head pain associated with a migraine headache.

If the subject is at risk of having a migraine, and the treatment is prophylactic, then an effective amount is an amount that reduces the risk of having a migraine headache. As used herein, an effective amount to reduce the risk of having a migraine headache is that amount which statistically reduces the number of subjects at risk of having a migraine headache who will go on to have a migraine, as minimally indicated by a severe or throbbing headache. In other words, the effective amount to reduce the risk of having a migraine is that amount which statistically inhibits or prevents the onset of head pain associated with having a migraine headache in subjects at risk.

According to the invention, agents which up-regulate EDG receptor signaling are useful for decreasing arterial blood flow and for inducing vasoconstriction. A subject who would benefit from decreased arterial blood flow is a subject who is experiencing inappropriate arterial blood flow, or who has a disorder associated with inappropriate arterial blood flow. An example of such a subject is one who is experiencing a migraine headache. In methods for decreasing arterial blood flow in a subject, the agent is administered to a subject in need of such treatment in an effective amount to decrease arterial blood flow. In methods for inducing vasoconstriction in a subject, the agent is administered to a subject in need of such treatment in an effective amount to induce vasoconstriction. A subject who would benefit from induced vasoconstriction or an increase in vasoconstriction is one who is experiencing inappropriate vasodilation or one who has a disorder associated with inappropriate vasodilation. An example of such a subject is one who is experiencing a migraine headache. Blood flow and vasoconstriction, particularly cerebral blood flow and vasoconstriction, are phenomenon which can be measured using conventional medical imaging techniques such as CT, MR, nuclear medicine and ultrasound, in some cases without a particular need for contrast agents. Thus, the amount of each individual agent necessary to decrease blood flow or to induce vasoconstriction, relative to baseline measurements for each parameter can be measured by administering doses of the particular agent to the subject, and observing the extent of change in either parameter relative to pre-administration measurements. In some embodiments, the arterial blood flow refers to cerebral artery blood flow (i.e., the blood flow into and through a cerebral artery). In other embodiments, vasoconstriction and vasodilation refers to cerebral vasoconstriction and cerebral vasodilation (i.e., the constriction and dilation of one or more cerebral arteries). As used herein, a cerebral artery includes but is not limited to an internal carotid artery, a middle cerebral artery, a posterior cerebral artery, a basilar artery, and a middle meningeal artery. All of the afore-mentioned arteries, in both their constricted or dilated states, and the blood flow through them, can be examined using in vivo imaging techniques.

EDG receptor signaling is known to stimulate the production and/or release of second messengers such as cAMP, intracellular Ca²⁺ and inositol triphosphate. Thus, agents which up-regulate EDG receptor signaling may be identified by the production and/or release of second messengers in cells or tissues expressing EDG receptors following the exposure to putative agonists. Putative agonists can be prescreened in vitro prior to in vivo studies for their ability to stimulate EDG receptor signaling.

According to yet other aspects of the invention, the subject having or at risk of having a migraine can also be administered, along with the agents of the invention, medicaments previously known to have some effect on migraine headaches and associated symptoms.

Examples of medicaments which can be used in combination with the agents of the invention in subjects having a migraine are sometimes referred to as abortive medicaments and these include OTC's such as aspirin-acetaminophen-caffeine combination; NSAIDS such as ibuprofen, diclofenac, ketoprofen, ketorolac, flurbiprofen, meclofenamate, naproxen sodium; glucocorticoids such as dexamethasone, prednisone, methylprednisone; acute abortives such as sumatripan, dihydroergotamine, ergotamine tartrate, isometheptene mucate-dichloralphenazone-acetaminophen combination, zolmitriptan, naratriptan and rizatriptan.

Examples of medicaments used in subjects at risk of having a migraine include beta-blockers such as propanolol, timolol, nadolol, metoprololc atenolol; calcium channel blockers such as verapamil, diltiazem, nicardipine, nifedipine, nimodipine; anti-epileptics such as divalproex sodium and neurontin; NSAIDS such as fenoprofen, flurbiprofen, ketoprofen, naproxen, nabumetone, oxaprozine; anti-depressants such as non-sedating tricyclics: protriptyline and desipramine, and sedating tricyclics: amitriptyline, doxepin, nortriptyline, imipramine; serotonin reuptake inhibitors such as fluoxetine, sertraline, paroxetine, nefazodone, venlafazine; and miscellaneous medicaments such as trazodone, bupropion, methylergonovine, phenobarbital-ergotamine tratrate-bellafoline combination, cyproheptadine, methysergide maleate, phenelzine (MAOI).

In yet another aspect, the invention provides methods and compositions to treat conditions which would benefit from, and which thus can be treated by, an inhibition of vasoconstriction or an increase in vasodilation. Such conditions can be categorized as cerebral occlusive disorders due to atherosclerosis, hyperlipidemia, and diabetes, and vascular dementia; cerebral ischemic disorders and disorders related to vasospasms resulting from traumatic brain injury, subarachnoid hemorrhage or other independent causes. Examples include but are not limited to stroke, subarachnoid hemorrhage and vasospasm. The invention provides related methods and compositions for increasing arterial blood flow in subjects who would benefit from increased arterial blood flow, as well as methods and compositions for inhibiting vasoconstriction in subjects who would benefit from an inhibition or vasoconstriction. In important embodiments, the vasoconstriction is vasoconstriction of cerebral arteries such as the basilar artery, the internal and external carotid arteries, the anterior cerebral artery, the middle cerebral artery, the posterior cerebral artery, the vertebral artery, the posterior inferior cerebellar artery and the middle meningeal artery. In preferred embodiments, the cerebral artery is a basilar artery or a middle cerebral artery.

Agents useful for increasing arterial blood flow, inhibiting vasoconstriction or inducing vasodilation are agents which down-regulate EDG receptor signaling. Agents which down-regulate EDG receptor signaling include sphingosine kinase inhibitors, sphingosine-1-phosphate phosphatase activators, and EDG receptor inhibitors. These agents embrace compounds which decrease the level of sphingosine kinase or EDG receptors, or increase the level of sphingosine-1-phosphate phosphatase. They also include compounds which interfere with EDG receptor signaling either by preventing EDG receptor binding to an agonist (e.g., a naturally occurring ligand), or interfering with a downstream factor required for EDG receptor signal transduction.

One important category of agents are those which inhibit sphingosine kinase activity, and thereby interfere with the production of sphingosine-1-phosphate. These latter compounds are herein referred to as sphingosine kinase antagonists. Some known sphingosine kinase antagonists are agents which molecularly mimic the natural substrates of sphingosinse kinase. Such antagonists bind to sphingosine kinase, in some instances irreversibly, and thereby prevent the binding of natural substrates of sphingosine kinase, ultimately preventing the phosphorylation of these substrates. Examples of sphingosine kinase antagonists include methylsphingosine, N,N-dimethyl sphingosine, trimethylsphingosine, D,L-threo-dihydrosphingosine and high density lipoprotein. Other sphingosine derivatives that can be used as sphingosine kinase inhibitors are described in U.S. Pat. Nos. 5,583,160; 5,627,171; 5,466,716; 5,391,800; 5,137,919; 5,151,360; 5,248,824; 5,260,288; and 5,331,014. De Jonghe et al. disclose the use of short-chain sphingoid bases, including short chain sphinganine analogs and 3-fluoro-sphingosine analogs as inhibitors of sphingosine kinase. (De Jonghe et al., Bioorg Med Chem Lett 1999 9 (21):3175-3180) The invention embraces the use of such sphingosine kinase antagonists provided they are useful in the treatment of conditions benefiting by inhibition of vasoconstriction or increased vasodilation. Other sphingosine kinase antagonists may bind sphingosine kinase at sites other than the substrate binding site, provided they ultimately interfere with the catalytic activity of the kinase. A suitable sphingosine kinase antagonist may interfere with the catalytic activity of sphingosine kinase by interfering with or preventing the interaction with substrates or catalysts, or interfering or preventing the release of products, or by preventing the modification of the substrates by the enzyme. The cloning of murine sphingosine kinase (GenBank Accession No. AF068748, AF068749) has been reported by Kohama et al., as have expression studies and activity studies aimed at measuring specific sphingosine kinase activity. (Kohama et al., J Biol Chem 1998 273 (37):23722-8) GenBank Accession Nos. NM_{—}021972 and XM_{—}012589 correspond to sequences of cloned human sphingosine kinase. Assays for any of the above agent classes have been described in the literature, and especially in PCT patent application Ser. No. PCT/AU98/00730 (WO 99/12533), the entire contents of which are incorporated herein by reference, which documents methods for measuring sphingosine kinase activity as well as methods for identifying sphingosine kinase agonists and antagonists.

Another important category of agents are sphingosine-1-phosphate phosphatase activators. Sphingosine-1-phosphate phosphatase activators are agents that increase the level of sphingosine-1-phosphate phosphatase activity. Such activators may do so by affecting the transcriptional, translational and/or post-translational mechanisms involved in sphingosine-1-phosphate phosphatase expression. Additionally, suitable activators may also affect the enzyme activity directly without affecting the expression level of the enzyme. This latter class of activators includes agents that bind to sphingosine-1-phosphate phosphatase and increase its activity and are referred to as sphingosine-1-phosphate phosphatase agonists.

Other agents which are useful according to the methods of the invention in the treatment of conditions which would benefit from inhibition of vasoconstriction include agents which interfere with EDG receptor expression at either the mRNA or protein level, or which interfere with EDG receptor interaction of other proteins, particularly if such interaction is necessary for signal transduction.

In accordance with the invention it was also discovered that sphingosine is an antagonist of sphingosine-1-phosphate induced vasoconstriction. Consistent with this observation is the idea that sphingosine can be used to inhibit vasoconstriction induced via EDG receptor. Thus, the methods of the invention also embrace the use of sphingosine to treat conditions which would benefit (i.e., can be treated) by increased vasodilation or inhibition of vasoconstriction such as, for example, stroke, subarachnoid hemorrhage and vasospasm. Activated platelets are known to release sphingosine-1-phosphate. As a result, activated platelets located at the site of hemorrhage would induce vasoconstriction, and thereby exacerbate the decreased blood flow to remaining regions of the brain. Administration of sphingosine would counter this vasoconstriction, allowing blood to flow throughout the cerebral vessels and reducing the ischemic damage which might otherwise occur.

Yet another category of useful agents in this regard is EDG receptor antagonists. As used herein, an EDG receptor antagonist is an agent which interferes with or prevents the transduction of a signal from an EDG receptor. An EDG receptor antagonist may interfere with the ability of an EDG receptor to bind an agonist. An antagonist may be an agent which competes with a naturally occurring ligand of EDG receptor for binding to the ligand binding site on the EDG receptor. Alternatively, the antagonist may bind to the EDG receptor at a site distinct from the ligand binding site, but in doing so, it may, for example, cause a conformational change in the receptor which is transduced to the ligand binding site, thereby precluding binding of the natural ligand. Alternatively, an EDG receptor antagonist may interfere with a component of the signaling cascade other than EDG receptor. This latter type of antagonist is referred to as a functional antagonist. A functional antagonist may interfere with intracellular signal transduction proteins, adaptors and secondary messengers such as, for example, protein kinase C or inositol triphosphate. In preferred embodiments, the EDG receptor antagonist is an EDG-1 receptor antagonist, EDG-3 receptor antagonist, EDG-5 receptor antagonist or EDG-8 receptor antagonist. In even more preferred embodiments, the EDG receptor antagonist is an EDG-3 receptor antagonist. In some embodiments, the agents of the invention act specifically on one member of the EDG receptor family. Therefore, in some embodiments, an EDG-3 receptor antagonist binds to EDG-3 receptor but not to any other EDG family member.

Yet another category of agents useful in methods relating to the inhibition of vasoconstriction or increased vasodilation are inhibitors of G proteins and Rho pathway and family members. G proteins are signaling molecules involved in transducing signals from EDG receptor following sphingosine-1-phosphate binding. G proteins include Gᵢ and Gₒ (which are involved in EDG-1 and EDG-8 signaling), and G_{q} and G_{12/13} (which are involved in EDG-3 and EDG-5 signaling). Inhibiting these proteins, particularly G_{q} and G_{12/13}, effectively inhibits the vasoconstrictive effects of sphingosine-1-phosphate, as described in the Examples. Compounds known to affect the Rho and Rho kinase pathway, and which can be used in the methods of the invention, include C3 exotoxin, C. Difficile toxin B, HA1077 (kinase inhibitor) and Y27632 (kinase inhibitor). Other G protein inhibitors are known in the art. Similarly, since sphingosine-1-phosphate signaling occurs at least in part through the extracellular signal-regulated kinase (ERK) pathway, members of this pathway, including MAPK, MAPKK, and MEK, can also be targeted for inhibition. It is to be understood that the invention similarly embraces activators of G proteins and ERK family members in the methods of the invention related to increased vasoconstriction and/or inhibition of vasodilation.

In another aspect, the present invention provides a method to treat a subject having or at risk of having a stroke. A stroke is the acute neurological injury resulting from a lack of oxygen to the brain which may result in reversible or irreversible paralysis, coma, speech problems and dementia. The injury is usually manifest as damage to nerve cells in the brain due to an interruption in blood flow, usually resulting from a blood clot or a blood vessel bursting. Approximately 80% of strokes are associated with cerebral ischemic infarction and 20% are associated with brain hemorrhage. A brain infarct typically increases in size during the acute period after ischemia begins, as some of the “penumbra” tissue dies. The infarct penumbra refers to tissue which is affected by the oxygen deficit from the vessel blockage or the hemorrhage, but which receives enough oxygen from other blood vessels to maintain temporary viability. The ultimate size of the infarct, and the resultant extent of neural damage to the stroke patient, are influenced by several factors, which form the basis of medical therapy for acute stroke. Current medical practice for the acute treatment of stroke includes anticoagulant and anti-platelet therapies.

As used herein, a subject having a stroke is treated by ameliorating, reducing or completely eliminating one or more symptoms associated with a stroke. As an example, a subject having a stroke is treated using the agents of the invention to reduce the extent of brain injury resulting from the stroke. The brain injury that follows a stroke, particularly an ischemic stroke, can be measured by determining an infarct size using standard medical imaging techniques. Accordingly, a reduction in the extent of brain injury is measured as a decrease in the infarct size. Likewise, functional tests measuring neurological deficits may provide further evidence of reduction in brain injury.

An important aspect of the invention is treatment of subjects who are having (i.e., experiencing) a stroke. If the subject is having a stroke, the treatment is preferably acute. Acute treatment for stroke subjects means administration of agents that down-regulate EDG receptor signaling (e.g., sphingosine kinase inhibitors, sphingosine-1-phosphate phosphatase activators, or EDG receptor inhibitors) at the onset of symptoms of the condition or at the onset of a substantial change in the symptoms of an existing condition. Sphingosine kinase antagonists, sphingosine-1-phosphate phosphatase agonists, or EDG receptor antagonists are preferred agents in some embodiments. In most subjects, administration of the treatment preferably is begun shortly after the initiation of the stroke, since early intervention will maximize the extent of potentially salvageable tissue. Treatment may be initiated, however, at any point in time prior to the completion of the infarction process, as assessed both on the basis of physical findings on neurological examination of the patient, as well as on the basis of imaging studies such as computed tomography or magnetic resonance imaging. Depending upon the embodiment, the severity of the vascular event, and the nature of the agent being administered (including potency, absorbance and clearance characteristics), the methods of the invention may be used to treat a patient within 2, 4, 6, 12, 24 or in some instances 36 hours after the onset of the event (e.g., stroke).

A subject having a stroke is so diagnosed by symptoms experienced and/or by a physical examination including interventional and non-interventional diagnostic tools such as CT and MR imaging. The methods of the invention are advantageous for the treatment of various clinical presentations of stroke subjects. A subject having a stroke may present with one or more of the following symptoms: paralysis, weakness, decreased sensation and/or vision, numbness, tingling, aphasia (e.g., inability to speak or slurred speech, difficulty reading or writing), agnosia (i.e., inability to recognize or identify sensory stimuli), loss of memory, co-ordination difficulties, lethargy, sleepiness or unconsciousness, lack of bladder or bowel control and cognitive decline (e.g., dementia, limited attention span, inability to concentrate). Using medical imaging techniques, it may be possible to identify a subject having a stroke as one having an infarct or one having hemorrhage in the brain.

Treatment of subjects at risk of having a stroke can include both acute and chronic treatments. Acute treatment in this regard refers to short term prophylactic treatment that is initiated before, concurrently with, or shortly after the initiation of the condition (or symptoms of the condition), or a procedure that may lead to the development of a stroke. Such conditions and procedures are described below. Chronic treatment of subjects at risk of having a stroke refers to long-term prophylactic treatment that may be administered to a subject at risk of having a stroke who is not presently experiencing a condition or a procedure that may lead to a stroke.

One aspect of the invention provides a method for the treatment of a subject at risk of having a stroke. As used herein, subjects at risk of having a stroke are a category determined according to conventional medical practice; such subjects may also be identified in conventional medical practice as having known risk factors for stroke or having increased risk of cerebrovascular events. Typically, the risk factors associated with cardiac disease are the same as are associated with stroke. The primary risk factors include hypertension, hypercholesterolemia, and smoking. In addition, atrial fibrillation, recent myocardial infarction and diabetes are important risk factors.

As used herein, subjects at risk of having a stroke also include individuals undergoing surgical or diagnostic procedures which risk release of emboli, lowering of blood pressure or decrease in blood flow to the brain, such as carotid endarterectomy, brain angiography, neurosurgical procedures in which blood vessels are compressed or occluded, cardiac catheterization, angioplasty, including balloon angioplasty, coronary by-pass surgery, or similar procedures.

Subjects at risk of having a stroke also include those who have experienced a brain injury or a myocardial infarction or those who have previously experienced a stroke. Subjects at risk of having a stroke also include individuals having any cardiac condition that may lead to decreased blood flow to the brain, such as atrial fibrillation, ventrical tachycardia, dilated cardiomyopathy and other cardiac conditions requiring anticoagulation. Subjects at risk of having a stroke also include individuals having conditions including arteriopathy or brain vasculitis, such as that caused by lupus, or congenital diseases of blood vessels, such as cerebral autosomal dominant arteriopathy with subcortical infarcts and leucoencephalopathy (CADASIL) syndrome. CADASIL syndrome is a disorder characterized by relapsing strokes with neuropsychiatric symptoms that affects relatively young adults of both sexes. CT scans have demonstrated occlusive cerebrovascular infarcts in the white matter, which is usually reduced.

Subjects at risk of having a stroke are also those suffering episodes of transient ischemic attacks. Transient ischemic attacks (TIA) which are often referred to an “mini-strokes,” result from the temporary or transient interruption of blood supply to an area of the brain. These disturbances usually lead to sudden and transient (e.g., 1-24 hours) diminution of brain activities and functions.

An important embodiment of the invention is treatment of a subject with an ischemic brain injury resulting from a stroke, a subarachnoid hemorrhage or a vasospasm. Ischemia is an acute condition associated with an inadequate flow of oxygenated blood to a tissue of the body, caused by the constriction or blockage of the blood vessels supplying it, therefore causing an ischemic injury to the particular tissue. Ischemia occurs any time that blood flow to a tissue is reduced below a critical level. This reduction in blood flow can result from: (i) the blockage of a vessel by an embolus (blood clot); (ii) the blockage of a vessel due to atherosclerosis; (iii) the breakage of a blood vessel (a bleeding stroke); (iv) the blockage of a blood vessel due to vasoconstriction such as occurs during vasospasms and possibly, during transient ischemic attacks (TIA) and following subarachnoid hemorrhage. Conditions in which ischemia occurs, further include (i) during myocardial infarction (when the heart stops, the flow of blood to organs is reduced and ischemia results); (ii) trauma; and (iii) during surgery (blood flow needs to be reduced or stopped to achieve the aims of surgery). When an ischemic event occurs, there is a gradation of injury that arises from the ischemic site. The cells at the site of blood flow restriction undergo necrosis and form the core of a lesion. A penumbra is formed around the core where the injury is not as immediately fatal but slowly progresses to cell death. In accordance with the invention, when ischemic subjects are administered an effective amount of an agent that down-regulates EDG receptor signaling, (including but not limited to a sphingosine kinase inhibitor such as a sphingosine kinase antagonist, and an EDG receptor inhibitor such as an EDG receptor antagonist, and a sphingosine-1-phosphate phosphatase activator such as a sphingosine-1-phosphate phosphatase agonist), progression to cell death is inhibited and the tissue is favorably affected. In certain important embodiments of the invention, the ischemic injury may be the result of a thrombotic event, stroke, pulmonary hypertension, arteriosclerosis, myocardial infarction, transplantation, organ reperfusion injury, chronic exposure to hypoxic conditions, homocystinuria, or CADASIL syndrome.

The therapeutic agents of the invention may be administered to subjects having a stroke in effective amounts to treat the stroke. An effective amount to treat a stroke is that amount necessary to ameliorate, reduce or eliminate altogether the brain injury or symptoms associate with stroke. Symptoms associated with stroke have been described above. Preferably, the effective amount to treat a stroke is that amount which reduces, or stabilizes an infarct size. As described in the Examples, using a rat embolic clot model, treatment with the sphingosine kinase inhibitor dimethylsphingosine (DMS) significantly reduced cerebral infarct size. Ideal agents are those which impact the greatest number of symptoms so that a subject begins functioning normally as soon as possible.

In yet other aspects of the invention, sphingosine kinase inhibitors, EDG receptor inhibitors or sphingosine-1-phosphate phosphatase activators, and preferably antagonists of sphingosine kinase or EDG receptors, or agonists of sphingosine-1-phosphate phosphatase, are administered to subjects at risk of having a stroke in effective amounts to reduce the risk of having a stroke. As used herein, an effective amount to reduce the risk of having a stroke is that amount which statistically reduces the number of subjects who go on to have a stroke from the pool of subjects at risk of having a stroke.

In yet other aspects of the invention relating to methods for increasing arterial blood flow, or alternatively inhibiting vasoconstriction, in a subject, the effective amount of an agent that down-regulates EDG receptor signaling (e.g., a sphingosine kinase inhibitor, a sphingosine-1-phosphate phosphatase activator, or an EDG receptor inhibitor) is that amount which increases blood flow, or which inhibits vasoconstriction, respectively. As mentioned above, blood flow and vasoconstriction are phenomena which are easily measured using conventional medical imaging techniques. A subject who would benefit from increased arterial blood flow is one who is experiencing reduced arterial blood flow due to, for example, vasoconstriction, vessel occlusion or hemorrhage. A subject who would benefit from an inhibition of vasoconstriction is one who is experiencing inappropriate vasoconstriction due to, for example, a vasospasm, and who may also be accumulating ischemic damage due to such inappropriate vasoconstriction.

The agents of the invention may be co-administered with other stroke therapeutic agents. The most common form of anti-stroke agents is the anti-platelet therapies which include aspirin, dipyridamole, sulfinpyrazone, clofibiate, ibuprofen, and ticlopidine.

In embodiments of the invention related to the increase in arterial blood flow or inhibition of vasoconstriction, a second agent may be co-administered to a subject with a condition treatable by the second agent in an amount effective to treat the condition, whereby the delivery of the second agent to a tissue of the subject, preferably the brain, is enhanced as a result of the increased blood flow from administering the first agent of the invention (e.g., a sphingosine kinase inhibitor, an EDG receptor inhibitor, or a sphingosine-1-phosphate phosphatase activator). The “second agent” may be any pharmacological compound or diagnostic agent, as desired. Preferred second agents are agents having a site of action in the brain. Such agents include adrenergic agent, amino acids, analeptic, analgesic, anesthetic, antagonists, antidote, anti-adrenergic agent, anti-anxiety agent, anti-cholinergic, anti-convulsant, anti-depressant, anti-emetic, anti-epileptic, anti-hypertensive, anti-fibrinolytic, anti-hyperlipidemia, anti-nauseant, anti-neoplastic (brain cancer), anti-obsessional agent, anti-obesity agent, anti-parkinsonian, anti-psychotic, appetite suppressant, blood glucose regulator, cognition adjuvant, cognition enhancer, dopaminergic agent, emetic, free oxygen radical scavenger, glucocorticoid, hypocholesterolemic, hypolipidemic, histamine H2 receptor antagonists, immunosuppressant, inhibitor, memory adjuvant, mental performance enhancer, mood regulator, mydriatic, neuromuscular blocking agent, neuroprotective, NMDA antagonist, post-stroke and post-head trauma treatment, psychotropic, sedative, sedative-hypnotic, serotonin inhibitor, tranquilizer, and treatment of cerebral ischemia, calcium channel blockers, free radical scavengers-antioxidants, GABA agonists, glutamate antagonists, AMPA antagonists, kainate antagonists, competitive and non-competitive NMDA antagonists, growth factors, opioid antagonists, phosphatidylcholine precursors, serotonin agonists, sodium- and calcium-channel blockers, and potassium channel openers.

The second agent may or may not be a medication for stroke, subarachnoid hemorrhage or vasospasm. In some embodiments, the subject is not having a stroke, a subarachnoid hemorrhage or a vasospasm and is not at risk of having a stroke, a subarachnoid hemorrhage or a vasospasm and the medication is not related to the therapeutic or prophylactic treatment of these disorders.

In addition to the foregoing brain-specific categories of agents, examples of categories of other pharmaceutical agents that can be used as second agents include: adrenocortical steroid; adrenocortical suppressant; alcohol deterrent; aldosterone antagonist; ammonia detoxicant; anabolic; analgesic; androgen; anorectic; anterior pituitary suppressant; anti-helmintic; anti-acne agent; anti-allergic; anti-amebic; anti-androgen; anti-anemic; anti-anginal; anti-arthritic; anti-asthmatic; anti-atherosclerotic; anti-bacterial; anti-cholelithic; anti-cholelithogenic; anti-coagulant; anti-coccidal; anti-diabetic; antidiarrheal; antidiuretic; anti-estrogen; anti-fungal; anti-glaucoma agent; anti-hemophilic; anti-hemorrhagic; anti-histamine; anti-hyperlipidemia; anti-hyperlipoproteinemic; anti-infective; anti-infective, topical; anti-inflammatory; anti-keratinizing agent; anti-malarial; anti-microbial; anti-mitotic; anti-mycotic, anti-neutropenic, anti-parasitic; anti-peristaltic, anti-pneumocystic; anti-proliferative; anti-prostatic hypertrophy; anti-protozoal; anti-pruritic; anti-rheumatic; anti-schistosomal; anti-seborrheic; anti-secretory; anti-spasmodic; anti-thrombotic; anti-tussive; anti-ulcerative; anti-urolithic; anti-viral; benign prostatic hyperplasia therapy agent; bone resorption inhibitor; bronchodilator; carbonic anhydrase inhibitor; cardiac depressant; cardioprotectant; cardiotonic; cardiovascular agent; choleretic; cholinergic; cholinergic agonist; cholinesterase deactivator; coccidiostat; depressant; diagnostic aid; diuretic; ectoparasiticide; enzyme inhibitor; estrogen; fibrinolytic; fluorescent agent; gastrointestinal motility effector; glucocorticoid; gonad-stimulating principle; hair growth stimulant; hemostatic; hormone; hypoglycemic; hypotensive; imaging agent; immunizing agent; immunomodulator; immunoregulator; immunostimulant; impotence therapy adjunct; keratolytic; LNRH agonist; liver disorder treatment; luteolysin; mucolytic; mucosal protective agent; nasal decongestant; neuroprotective; non-hormonal sterol derivative; oxytocic; plasminogen activator; platelet activating factor antagonist; platelet aggregation inhibitor; potentiator; progestin; prostaglandin; prostate growth inhibitor; prothyrotropin; pulmonary surface; radioactive agent; regulator; relaxant; repartitioning agent; scabicide; sclerosing agent; selective adenosine Al antagonist; serotonin receptor antagonist; steroid; stimulant; suppressant; symptomatic multiple sclerosis; synergist; thyroid hormone; thyroid inhibitor; thyromimetic; treatment of amyotrophic lateral sclerosis; treatment of Paget's disease; treatment of unstable angina; uricosuric; vasoconstrictor; vasodilator; vulnerary; wound healing agent; xanthine oxidase inhibitor. In an important embodiment, the second therapeutic agent is TPA.

A further aspect of the invention provides a method for the treatment of a subject having or at risk of having a subarachnoid hemorrhage. A subarachnoid hemorrhage is an acute condition involving sudden hemorrhage into the space between the arachnoid membrane and the pia mater (adjacent to the brain). The subarachnoid is the layer of tissue between the arachnoid membrane and the pia mater, which contains cerebrospinal fluid (CSF). Subarachnoid hemorrhage is often secondary to a head injury or a blood vessel defect known as an aneurysm. In some instances, subarachnoid hemorrhage can induce a cerebral vasospasm that in turn leads to an ischemic stroke. A common manifestation of a subarachnoid hemorrhage is the presence of blood in the CSF.

Subjects having a subarachnoid hemorrhage can be identified by a number of symptoms. For example, a subject having a subarachnoid hemorrhage will present with blood in the subarachnoid, usually in a large amount. Subjects having a subarachnoid hemorrhage can also be identified by an intracranial pressure that approximates mean arterial pressure, by a fall in cerebral perfusion pressure or by the sudden transient loss of consciousness (sometimes preceded by a painful headache). In about half of cases, subjects present with a severe headache which may be associated with physical exertion. Other symptoms associated with subarachnoid hemorrhage include nausea, vomiting, memory loss, hemiparesis and aphasia. Subjects having a subarachnoid hemorrhage can also be identified by the presence of creatine kinase-BB isoenzyme activity in their CSF. This enzyme is enriched in the brain but is normally not present in the CSF. Thus, its presence in the CSF is indicative of “leak” from the brain into the subarachnoid. Assay of creatine-kinase BB isoenzyme activity in the CSF is described by Coplin et al. (Coplin, et al, Arch Neurol, 1999, 56(11):1348-1352) Additionally, a spinal tap or lumbar puncture can be used to demonstrate if there is blood present in the CSF, a strong indication of a subarachnoid hemorrhage. A cranial CT scan or an MRI can also be used to identify blood in the subarachnoid region. Angiography can also be used to determine not only whether a hemorrhage has occurred but also the location of the hemorrhage.

Subarachnoid hemorrhage commonly results from rupture of an intracranial saccular aneurysm or from malformation of the arteriovenous system in, and leading to, the brain. Accordingly, a subject at risk of having a subarachnoid hemorrhage includes subjects having a saccular aneurysm as well as subjects having a malformation of the arteriovenous system. It is estimated that 5% of the population have such aneurysms yet only 1 in 10,000 people actually have a subarachnoid hemorrhage. The top of the basilar artery and the junction of the basilar artery with the superior cerebellar or the anterior inferior cerebellar artery are common sites of saccular aneurysms. Subjects having a subarachnoid hemorrhage may be identified by an eye examination, whereby slowed eye movement may indicate brain damage. A subject with a developing saccular aneurysm can be identified through routine medical imaging techniques, such as CT and MRI. A developing aneurysm forms a mushroom-like shape (sometimes referred to as “a dome with a neck” shape).

Subjects at risk of having a subarachnoid hemorrhage also include subjects having disorders associated with aneurysm or weakened blood vessels, including a history of polycystic kidney disease; fibromuscular dysplasia; aneurysms in other blood vessels; and high blood pressure. Subjects at risk of having a subarachnoid hemorrhage via a rupture of a intracranial saccular aneurysm commonly manifest prodromal symptoms. These include dilation of the pupil, loss of light reflex, pain above and behind the eye, pain in and behind the eye and in the low temple and sudden unexplained headaches. In some subjects, small amounts of blood, called “warning leaks” may prematurely seep out of the vasculature. This leaking may be intermittent. Subjects having such leaks, as evidenced by the presence of small amounts of blood in the subarachnoid (using a CT or MR scan), are also subjects at risk of having a subarachnoid hemorrhage. A lumbar puncture may also be performed to detect blood in the subarachnoid. This latter method may be preferable when the suspected leaks are too small to be visualized using CT. Subjects who test positive for blood in the subarachnoid are considered to be subjects either having a subarachnoid hemorrhage (particularly if the amount of blood is large and if other subarachnoid hemorrhage symptoms are experienced) or at risk of having a subarachnoid hemorrhage. Subjects at risk of having a subarachnoid hemorrhage also include those who are experiencing or who have experienced venous or sinus thrombosis, brain tumors, CNS tumors, spinal or cerebral dural or parenchymal vascular malformation, and rupture of small superficial arteries.

According to some aspects of the invention, agents which down-regulate EDG receptor signaling (e.g., sphingosine kinase inhibitors, sphingosine-1-phosphate phosphatase activators, or EDG receptor inhibitors) are administered to subjects having a subarachnoid hemorrhage in an effective amount to treat the subarachnoid hemorrhage. As used herein, an effective amount to treat a subarachnoid hemorrhage is that amount which ameliorates, reduces or eliminates altogether the pain associated with a subarachnoid hemorrhage and reduces or stabilizes the size of an infarct that results from the hemorrhage. An ideal agent would affect the greatest number of symptoms.

If the agent is administered in a prophylactic mode, it is administered to a subject at risk of having a subarachnoid hemorrhage in an effective amount to reduce the risk of having a subarachnoid hemorrhage. An effective amount which reduces the risk of having a subarachnoid hemorrhage is that amount which reduces the number of subjects who will ultimately go on to experience a subarachnoid hemorrhage from the pool of subjects who are at risk of having a subarachnoid hemorrhage. In other words, an effective amount which reduces the risk of having a subarachnoid hemorrhage is that amount which statistically prevents or inhibits the onset of symptoms associated with a subarachnoid hemorrhage in subjects at risk.

The agents of the invention can be administered with agents which are normally administered to subjects having subarachnoid hemorrhage. Such agents include acetaminophen, meperidine, phenobarbitol or other sedatives.

According to yet another aspect of the invention, a method is provided for the treatment of a subject having or at risk of having a vasospasm. A vasospasm is a sudden decrease in the internal diameter of a blood vessel that results from contraction of smooth muscle within the wall of the vessel. Vasospasms result in decreased blood flow, but increased system vascular resistance. It is generally believed that vasospasm is caused by local injury to vessels, such as that which results from atherosclerosis and other structural injury including traumatic head injury. Cerebral vasospasm is a naturally occurring vasoconstriction which can also be triggered by the presence of blood in the CSF, a common occurrence after rupture of an aneurysm or following traumatic head injury. Cerebral vasospasm can ultimately lead to brain cell damage, in the form of cerebral ischemia and infarction, due to interrupted blood supply.

A subject having a vasospasm is a subject who presents with diagnostic markers and symptoms associated with vasospasm. Diagnostic markers include the presence of blood in the CSF and/or a recent history of a subarachnoid hemorrhage. Vasospasm associated symptoms include paralysis on one side of the body, inability to vocalize the words or to understand spoken or written words, and inability to perform tasks requiring spatial analysis. Such symptoms may develop over a few days, or they may fluctuate in their appearance, or they may present abruptly.

MR angiography and CT angiography can be used to diagnose cerebral vasospasm. Angiography is a technique in which a contrast agent is introduced into the blood stream in order to view blood flow and/or arteries. A contrast agent is required because blood flow and/or arteries are sometimes only weakly apparent in a regular MR or CT scan. Appropriate contrast agents will vary depending upon the imaging technique used. For example, gadolinium is a common contrast agent used in MR scans. Other MR appropriate contrast agents are known in the art. Transcranial Doppler ultrasound can also be used to diagnose and monitor the progression of a vasospasm. As mentioned earlier, the presence of blood in the cerebrospinal fluid can be detected using CT scans. However, in some instances where the amount of blood is so small as to not be detected by CT, a lumbar puncture is warranted.

A subject at risk of a vasospasm includes a subject who has detectable blood in the cerebrospinal fluid, or one who has a detectable aneurysm as detected by a CT scan, yet has not begun to experience the symptoms associated with having a vasospasm. A subject at risk of a vasospasm is also one who has experienced a traumatic head injury, independent of a subsequent subarachnoid hemorrhage. Traumatic head injury usually results from a physical force to the head region, in the form of a fall or a forceful contact with a solid object. Subjects at risk of vasospasm include subjects diagnosed with conditions regarded as high risk of cerebrovascular disease. These include hypertension, smoking, diabetes mellitus, alcohol use, cardiovascular disease and drug abuse. Subjects at risk of a vasospasm include those who have recently (e.g., in the last two weeks) experienced a subarachnoid hemorrhage. Subjects at risk of having a vasospasm also include those who present with a subarachnoid hemorrhage. Subjects at risk of having a vasospasm also include subjects who will undergo angiography.

In one aspect of the invention, agents which down-regulate EDG receptor signaling (e.g., a sphingosine kinase inhibitor, a sphingosine-1-phosphate phosphatase activator, or an EDG receptor inhibitor) is administered to the subject having a vasospasm in an effective amount to treat a vasospasm. An effective amount to treat a vasospasm may be that amount necessary to ameliorate, reduce or eliminate altogether one or more symptoms relating to a vasospasm, preferably including brain damage that results from vasospasm such as an infarct. Brain damage can be measured anatomically using medical imaging techniques to measure infarct sizes. Alternatively or in conjunction, brain damage may be measured functionally in terms of cognitive or sensory skills of the subject.

Subjects at risk of vasospasm are currently administered a variety of preventative medications including calcium channel blockers (e.g., nimodipine), phenylephrine, dopamine, as well as a combination of mannitol and hyperventilation. Some forms of prophylactic treatments aim to increase the cerebral perfusion pressure. In accordance with the present invention, any of these prophylactic therapies may be co-administered to a subject at risk of having a vasospasm along with the agents of the invention.

Yet another condition which can be treated with the agents of the invention which inhibit vasoconstriction or which promote vasodilation, namely sphingosine kinase inhibitors, sphingosine-1-phosphate phosphatase activators and EDG receptor inhibitors, is transient ischemia attacks (TIA). TIA are the manifestation of a brain disorder caused by temporary disturbance of the blood supply to an area of the brain, resulting in a sudden, brief decrease in brain functions. TIA are usually short-lived, lasting roughly 5 to 30 minutes, yet can reoccur several times in a day.

Subjects having TIA are identified by a number of symptoms including changes in vision, changes in speech, decreased movement or sensation, and changes in consciousness. Other symptoms include tingling and/or numbness, weakness, vertigo and lack of co-ordination. The symptoms generally appear abruptly and last anywhere from 1 hour to 1 day. Subjects having TIA are diagnosed as such using neurologic examination, including examination of the eye, and measurement of eye pressure. Other diagnostic tests which can be used to identify a subject having a TIA include cranial CT and MR scans, ultrasound of the carotid duplex, and cerebral arteriogram. Laboratory tests which can be used to identify a subject having TIA include measuring blood glucose, general blood chemistry, and serum lipids.

Subjects at risk of having TIA include subjects who have a conditions such as atherosclerosis, blood disorders such as polycythemia, sickle cell anemia and hyperviscosity syndromes in which the blood is thicker than normal. Subjects at risk of TIA are also those who have experienced spasm of the small arteries in the brain, those who have abnormalities of blood vessels caused by disorders such as fibromuscular dysplasia, inflammation of the arteries (arteritis, polyarteritis, and granulomatous angiitis), systemic lupus erythematosus, and syphillis. In subjects with a pre-existing vascular lesion, hypotension can lead to a stroke. Other risk factors for TIA include smoking, high blood pressure, heart disease, diabetes mellitus and increasing age. TIA are more common in men.

Yet another disorder which can be treated with agents that down-regulate EDG receptor signaling is granulomatous arteritis (GA), in which the internal diameter of extracranial, but also intracranial arteries, decreases. GA can be manifest by ocular symptoms including sudden loss of vision in one eye, and it usually strikes older subjects.

In yet another aspect, the invention provides methods and compositions for treating peripheral vasoconstriction, particularly abnormal peripheral vasoconstriction such as that which occurs in hypertensive subjects. Agents of the invention, namely agents which down-regulate EDG receptor signaling (e.g., EDG-3 receptor antagonists) may be administered to hypertensive subjects in order to inhibit inappropriate vasoconstriction.

As described earlier, the agents of the invention, namely agents which up-regulate or down-regulate EDG receptor signaling are administered in effective amounts. In general, an effective amount is any amount that can cause a beneficial change in a desired tissue. Preferably, an effective amount is that amount sufficient to cause a favorable phenotypic change in a particular condition such as a lessening, alleviation or elimination of a symptom or of a condition as a whole.

In general, an effective amount is that amount of a pharmaceutical preparation that alone, or together with further doses, produces the desired response. This may involve only slowing the progression of the disease temporarily, although more preferably, it involves halting the progression of the disease permanently or delaying the onset of or preventing the disease or condition from occurring. This can be monitored by routine methods. Generally, doses of active compounds would be from about 0.01 mg/kg per day to 1000 mg/kg per day. It is expected that doses ranging from 50-500 mg/kg will be suitable, preferably orally and in one or several administrations per day.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. Lower doses will result from certain forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds. It is preferred generally that a maximum dose be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The agents of the invention, including but not limited to sphingosine kinase activators and inhibitors, sphingosine-1-phosphate phosphatase activators and inhibitors, and the EDG receptor activators and inhibitors, may be combined, optionally, with a pharmaceutically-acceptable carrier to form a pharmaceutical preparation. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy. In some aspects, the pharmaceutical preparations comprise an agent of the invention in an amount effective to treat a disorder. In other aspects, the pharmaceutical preparation comprises the agent of the invention, one or more of the second therapeutic agents discussed herein, and a pharmaceutically acceptable carrier. The nature of the second therapeutic agent will depend upon the condition of the subject being treated. As an example of this latter aspect, a pharmaceutical preparation may contain an agent that down-regulates EDG receptor signaling (e.g., a sphingosine kinase antagonist such as suramin) and a psychiatric agent (e.g., an anti-depressant agent such as fluoxetine (Prozac™). As well, such combinations may be provided together in a kit, either commingled or in separate containers.

The pharmaceutical preparations may contain suitable buffering agents, including: acetic acid in a salt; citric acid in a salt; boric acid in a salt; and phosphoric acid in a salt. The pharmaceutical compositions also may contain, optionally, suitable preservatives, such as: benzalkonium chloride; chlorobutanol; parabens and thimerosal.

A variety of administration routes are available. The particular mode selected will depend, of course, upon the particular drug selected, the severity of the condition being treated and the dosage required for therapeutic efficacy. The methods of the invention, generally speaking, may be practiced using any mode of administration that is medically acceptable, meaning any mode that produces effective levels of the active compounds without causing clinically unacceptable adverse effects. Such modes of administration include oral, rectal, topical, nasal, interdermal, or parenteral routes. The term “parenteral” includes subcutaneous, intravenous, intramuscular, or infusion. Intravenous or intramuscular routes are not particularly suitable for long-term therapy and prophylaxis. As an example, pharmaceutical compositions for the acute treatment of subjects having a migraine headache may be formulated in a variety of different ways and for a variety of administration modes including tablets, capsules, powders, suppositories, injections and nasal sprays.

The pharmaceutical preparations may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as a syrup, elixir or an emulsion.

Compositions suitable for parenteral administration conveniently comprise a sterile aqueous preparation of the agents of the invention, which is preferably isotonic with the blood of the recipient. This aqueous preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono-or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

Other delivery systems can include time-release, delayed release or sustained release delivery systems. Such systems can avoid repeated administrations of the active compound, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di-and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the active compound is contained in a form within a matrix such as those described in U.S. Pat. Nos. 4,452,775, 4,675,189 and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

Use of a long-term sustained release implant may be desirable. Long-term release, are used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

The methods of the invention further embrace the identification of activators and inhibitors, preferably agonist and antagonists, of sphingosine kinase, sphingosine-1-phosphate phosphatase, and EDG receptors.

Putative compounds can be synthesized from peptides or other biomolecules including but not limited to saccharides, fatty acids, sterols, isoprenoids, purines, pyrimidines, derivatives or structural analogs of the above, or combinations thereof and the like. Phage display libraries and chemical combinatorial libraries can be used to develop and select synthetic compounds which are capable of activating or inhibiting sphingosine kinase, sphingosine-1-phosphate phosphatase, or EDG receptors. Also envisioned in the invention is the use of compounds made from peptoids, random bio-oligomers (U.S. Pat. No. 5,650,489), benzodiazepines, diversomeres such as dydantoins, benzodiazepines and dipeptides, nonpeptidal peptidomimetics with a beta-D-glucose scaffolding, oligocarbamates or peptidyl phosphonates.

In one aspect, the invention envisions the use of library technology to identify small molecules, including small peptides, which bind to a EDG receptor ligand binding site (e.g., an EDG-3 receptor ligand binding site), or a protein interaction domain of an EDG receptor (e.g., a G protein interacting domain), or a sphingosine kinase or a sphingosine-1-phosphate phosphatase substrate binding, or a sphingosine kinase or a sphingosine-1-phosphate phosphatase catalytic site. One advantage of using libraries for agonist or antagonist identification is the facile manipulation of millions of different putative candidates of small size in small reaction volumes (i.e., in synthesis and screening reactions). Another advantage of libraries is the ability to synthesize agonists or antagonists which might not otherwise be attainable using naturally occurring sources, particularly in the case of non-peptide moieties.

Many if not all of these compounds can be synthesized using recombinant or chemical libraries. A vast array of candidate compounds can be generated from libraries of synthetic or natural compounds. Libraries of natural compounds in the form of bacterial, fungal, plant and animal extracts are available or can readily produced. Natural and synthetically produced libraries and compounds can be readily modified through conventional chemical, physical, and biochemical means. In addition, compounds known to bind to and thereby act as agonists or antagonists of sphingosine kinase, sphingosine-1-phosphate phosphatase, or EDG receptors, particularly EDG-3 receptor, may be subjected to directed or random chemical modifications such as acylation, alkylation, esterification, amidification, etc. to produce structural analogs which may function similarly or perhaps with greater specificity.

Small molecule combinatorial libraries may also be generated. A combinatorial library of small organic compounds is a collection of closely related analogs that differ from each other in one or more points of diversity and are synthesized by organic techniques using multi-step processes. As an example, analogs of sphingosine can be generated which function as EDG receptor antagonists. As another example and as mentioned earlier, De Jonghe and co-workers have identified a number of short-chain sphingoid bases, including 3-fluoro-sphinganines, which function as sphingosine kinase inhibitors. Thus, analogs of these latter category of compounds can be synthesized using the combinatorial libraries taught herein.

Combinatorial libraries include a vast number of small organic compounds. One type of combinatorial library is prepared by means of parallel synthesis methods to produce a compound array. A “compound array” as used herein is a collection of compounds identifiable by their spatial addresses in Cartesian coordinates and arranged such that each compound has a common molecular core and one or more variable structural diversity elements. The compounds in such a compound array are produced in parallel in separate reaction vessels, with each compound identified and tracked by its spatial address. Examples of parallel synthesis mixtures and parallel synthesis methods are provided in PCT published patent application WO 95/18972, published Jul. 13, 1995 and U.S. Pat. No. 5,712,171 granted Jan. 27, 1998 and its corresponding PCT published patent application WO 96/22529, which are hereby incorporated by reference.

The compounds generated using the recombinant and chemical libraries described herein can be initially screened to identify putative compounds by virtue of their ability to bind to sphingosine kinase, sphingosine-1-phosphate phosphatase, or EDG receptors, preferably EDG-3 receptors. Compounds such as library members can be screened for their ability to bind to sphingosine kinase, sphingosine-1-phosphate phosphatase, or EDG receptors in vitro using standard binding assays well known to the ordinary artisan and described below. For binding to an EDG receptor, the EDG receptor may be presented in a number of ways including but not limited to cells expressing the EDG receptor of interest, an isolated extracellular domain of an EDG receptor, a fragment thereof or a fusion protein of the extracellular domain of an EDG receptor and another protein such as an immunoglobulin or a GST polypeptide. For some high throughput screening assays the use of purified forms of an EDG receptor, its extracellular domain or a fusion of its extracellular domain with another protein may be preferable. Isolation of binding partners may be performed in solution or in solid state according to well-known methods. For binding to a sphingosine kinase or sphingosine-1-phosphate phosphatase, the sphingosine kinase may be presented in a purified (e.g., a recombinantly produced form), or in the form of a cell lysate from cells known to express sphingosine kinase or sphingosine-1-phosphate phosphatase, either naturally or as a result of transfection of a vector encoding sphingosine kinase or sphingosine-1-phosphate phosphatase. In these latter screens, it may be desirable to pre-screen a library of molecules by exposure to a cell or a cell lysate which does not express sphingosine kinase or sphingosine-1-phosphate phosphatase (i.e., the non-transfected parent cell) in order to deplete the population of library members that are not of interest.

Standard binding assays are well known in the art, and a number of these are suitable in the present invention including ELISA, competition binding assay (particularly suitable in the present invention since some native binding partners of sphingosine kinase, sphingosine-1-phosphate phosphatase, and EDG receptors are known), sandwich assays, radioreceptor assays using radioactively labeled ligands or substrates of EDG receptors, sphingosine-1-phosphate phosphatase, and sphingosine kinase (with the binding of the native, radioactively labeled, ligand or substrate being competed with by the putative agonist or antagonist), electrophoretic mobility shift assays, immunoassays, cell-based assays such as two- or three-hybrid screens, etc. The nature of the assay is not essential provided it is sufficiently sensitive to detect binding of a small number of library members.

A variety of other reagents also can be included in the binding mixture. These include reagents such as salts, buffers, neutral proteins (e.g., albumin), detergents, etc. which may be used to facilitate optimal enzyme-substrate or receptor-ligand interactions. Such a reagent may also reduce non-specific or background interactions of the reaction components. Other reagents that improve the efficiency of the assay may also be used. The mixture of the foregoing assay materials is incubated under conditions under which the EDG receptor or the sphingosine-1-phosphate phosphatase normally specifically binds one or more of its native ligands (e.g., sphingosine-1-phosphate), or which sphingosine kinase specifically binds one or more of its substrates (e.g., sphingosine). The order of addition of components, incubation temperature, time of incubation, and other perimeters of the assay may be readily determined. Such experimentation merely involves optimization of the assay parameters, not the fundamental composition of the assay. Incubation temperatures typically are between 4° C. and 40° C. Incubation times preferably are minimized to facilitate rapid, high throughput screening, and typically are between 0.1 and 10 hours. After incubation, the presence or absence of specific binding between the EDG receptor and sphingosine-1-phosphate or the compound being screened, or the presence or absence of specific binding between sphingosine kinase and sphingosine or the compound being screened, or the presence or absence of the specific binding between sphingosine-1-phosphate phosphatase and sphingosine-1-phosphate is detected by any convenient method available to the user.

Typically, a plurality of assay mixtures are run in parallel with different agent concentrations to obtain a different response to the various concentrations. One of these concentrations serves as a negative control, i.e., at zero concentration of agent or at a concentration of agent below the limits of assay detection.

For cell-free binding type assays, a separation step is often used to separate bound from unbound components. The separation step may be accomplished in a variety of ways. Conveniently, at least one of the components is immobilized on a solid substrate, from which the unbound components may be easily separated. The solid substrate can be made of a wide variety of materials and in a wide variety of shapes, e.g., columns or gels of polyacrylamide, agarose or sepharose, microtiter plates, microbeads, resin particles, etc. The substrate preferably is chosen to maximum signal to noise ratios, primarily to minimize background binding. The separation step may include rinses or washes.

One or more of the components usually comprises, or is coupled to, a detectable label. A wide variety of labels can be used, such as those that provide direct detection (e.g., radioactivity, luminescence, optical or electron density, etc). or indirect detection (e.g., epitope tag such as the FLAG epitope, enzyme tag such as horseradish peroxidase, etc.). The label may be bound to a library member, or incorporated into the structure of the library member. The EDG receptor, its ligand and the putative activating or inhibiting compound of the invention (and likewise, sphingosine kinase, its substrate and the putative activating or inhibiting compound of the invention, and sphingosine-1-phosphate phosphatase, its substrate and the putative activating or inhibiting compound of the invention) may be labeled by a variety of means for use in screening. There are many different labels and methods of labeling known to those of ordinary skill in the art. Examples of the types of labels which can be used in the present invention include enzymes, radioisotopes, fluorescent compounds, colloidal metals, chemiluminescent compounds, and bioluminescent compounds. In one important embodiment, the label is a radioisotope which is incorporated into the compound during synthesis (e.g., P³², C¹⁴ or H³ can each be used to replace a naturally occurring phosphorus, carbon or hydrogen atom in the structure). Preferably, all the compound synthesized will share the common radioisotope replacement which might conveniently occur in a backbone region of the analog. Those of ordinary skill in the art will know of other suitable labels for binding to the binding partners used in the screening assays, or will be able to ascertain such, using routine experimentation. Furthermore, the coupling of these labels the binding partners used in the screening assays of the invention can be done using standard techniques common to those of ordinary skill in the art.

Another labeling technique which may result in greater sensitivity consists of coupling the binding partners to low molecular weight haptens. These haptens can then be specifically altered by means of a second reaction. For example, it is common to use haptens such as biotin, which reacts with avidin, or dinitrophenol, pyridoxal, or fluorescein, which can react with specific anti-hapten antibodies.

A variety of methods may be used to detect the label, depending on the nature of the label and other assay components. For example, the label may be detected while bound to the solid substrate or subsequent to separation from the solid substrate. Labels may be directly detected through optical or electron density, radioactive emissions, nonradiative energy transfers, etc. or indirectly detected with antibody conjugates, streptavidin-biotin conjugates, etc. Methods for detecting the labels are well known in the art.

Once compounds have been identified which are capable of binding either sphingosine kinase, sphingosine-1-phosphate phosphatase, or EDG receptors, these compounds can be further screened for their ability to modulate vasoconstriction. An exemplary assay for measuring effect of a compound on vasoconstriction the recording of isometric tension in isolated blood vessels which is described in the Examples. In that assay, rat arteries including basilar, carotid, coronary and femoral arteries, are harvested and bathed in a physiological solution and then mounted onto a myograph. The identical arterial segments may be used to test the contractile response to a control substance (such as for example, potassium chloride) and the test compound (e.g., compounds which are identified through their ability to bind to sphingosine kinase, sphingosine-1-phosphate phosphatase or EDG receptors). Contractile responses to agonists of either sphingosine kinase, sphingosine-1-phosphate phosphatase, or EDG receptors can be measured directly using the contraction achieved using KCl or sphingosine-1-phosphate as a reference. The arterial segments may be exposed to increasing amounts of the test compound in order to arrive at a dose-response curve and an estimation of the appropriate dosage. Modulation of vasoconstriction can also be measured using an assay which records intraluminal pressure in perfused isolated vessels.

For measuring the antagonistic potential of other substances, the arterial segments may be pre-incubated with the test compound (i.e., putative antagonist) followed by incubation with a known agonist such as sphingosine-1-phosphate. The contractile response achieved with sphingosine-1-phosphate with and without preincubation with the test compound will determine whether the compound has any antagonistic activity. In an alternative approach, the arterial segments may be exposed to the test compound and the known agonist at the same time. The contractile response can then be compared to the response in the presence of the agonist alone or the agonist and a control solution. If the contractile response is decreased in the presence of the test compound, then this indicates that the test compound is capable of antagonizing agonist-induced vasoconstriction.

Other methods for identifying EDG receptor agonists or antagonists are described in PCT patent application WO 99/35259 and PCT patent application WO 99/46277, the contents of both of which are incorporated by reference herein in their entirety.

In yet another screening method, compounds which bind to sphingosine kinase can be tested for their agonist or antagonist activity using methods known in the art. PCT patent application WO 99/12533 describes a method for identifying agonists or antagonists of sphingosine kinase using radiolabeled ATP (e.g., ³²P or ³³P) as a source of detectable label, and sphingosine as the substrate. Accumulation of radiolabeled sphingosine phosphate is used as the readout. Sphingosine kinase can be incubated with the test compound prior to or simultaneously with sphingosine and ATP substrates. An appropriate control may be the readout in the absence of the test compound or in a control solution lacking only the test compound. An increase or a decrease in the amount of radiolabeled sphingosine phosphate produced in the presence of the test compound relative to the control solution is indicative of a sphingosine kinase agonist and antagonist, respectively. The entire contents of PCT patent application WO 99/12533 are herein incorporated by reference.

In a further screening method, compounds that bind to sphingosine-1-phosphate phosphatase can be tested for their agonist or antagonist activity using methods known in the art. An exemplary sphingosine-1-phosphate phosphatase assay is described by Mandala et al. (Mandala et al., PNAS, 97:7859-7864, 2000) Briefly, the assay involves transfecting cells (e.g., NIH 3T3 fibroblasts ATCC CRL-1658 or human embryonic kidney cells HEK ATCC CRL-1573) with a sphingosine-1-phosphate phosphatase encoding plasmid (e.g., pcDNA3.1 with the murine sphingosine-1-phosphate phosphatase coding sequence from GenBank accession number AF247177 cloned into it). Forty-eight hours after transfection, the transfected cells are harvested and lysed by a series of freeze-thaw cycles. Homogenized membrane fractions are then incubated with ³²P labeled sphingosine-1-phosphate (Kohama, et al., J. Biol. Chem., 273:23722-23728, 1998) for 30 minutes at 37° C. Following the incubation, ³²P labeled sphingosine-1-phosphate is extracted and quantitated by thin layer chromatography (Mandala, et al., PNAS, 95:150-155, 1998). Sphingosine-1-phosphate phosphatase activity can be represented by the amount of ³²P released from ³²P labeled sphingosine-1-phosphate during incubation with sphingosine-1-phosphate phosphatase. Another exemplary method is described by Brindley et al., Methods in Enzymology, 311:233-244, 1999.

Similar methods can be employed for the purpose of identifying G protein inhibitors and inhibitors of the ERK signaling pathway.

As mentioned above, the invention embraces antisense oligonucleotides that selectively bind to a nucleic acid molecules encoding an EDG receptor, a sphingosine kinase or a sphingosine-1-phosphate phosphatase, to decrease expression and activity of each of these proteins.

As used herein, the term “antisense oligonucleotide” or “antisense” describes an oligonucleotide that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA. The antisense molecules are designed so as to interfere with transcription or translation of a target gene upon hybridization with the target gene or transcript. Antisense oligonucleotides that selectively bind to either a nucleic acid molecule encoding an EDG receptor (preferably an EDG-3 receptor), a sphingosine kinase or a sphingosine-1-phosphate phosphatase are particularly preferred. Those skilled in the art will recognize that the exact length of the antisense oligonucleotide and its degree of complementarity with its target will depend upon the specific target selected, including the sequence of the target and the particular bases which comprise that sequence.

It is preferred that the antisense oligonucleotide be constructed and arranged so as to bind selectively with the target under physiological conditions, i.e., to hybridize substantially more to the target sequence than to any other sequence in the target cell under physiological conditions. Based upon the nucleotide sequences of nucleic acid molecules encoding EDG receptor, sphingosine kinase or sphingosine-1-phosphate phosphatase (e.g., GenBank Accession Nos. NM-005226, X83864, and AF184914 for EDG-3 receptor; AF068748, AF068749, NM_{—}021972 and XM_{—}012589 for sphingosine kinase; AF247177 for sphingosine-1-phosphate phosphatase) or upon allelic or homologous genomic and/or cDNA sequences, one of skill in the art can easily choose and synthesize any of a number of appropriate antisense molecules for use in accordance with the present invention. In order to be sufficiently selective and potent for inhibition, such antisense oligonucleotides should comprise at least about 10 and, more preferably, at least about 15 consecutive bases which are complementary to the target, although in certain cases modified oligonucleotides as short as 7 bases in length have been used successfully as antisense oligonucleotides. See Wagner et al., Nat. Med. 1(11):1116-1118, 1995. Most preferably, the antisense oligonucleotides comprise a complementary sequence of 20-30 bases. Although oligonucleotides may be chosen which are antisense to any region of the gene or mRNA transcripts, in preferred embodiments the antisense oligonucleotides correspond to N-terminal or 5′ upstream sites such as translation initiation, transcription initiation or promoter sites. In addition, 3′-untranslated regions may be targeted by antisense oligonucleotides. Targeting to mRNA splicing sites has also been used in the art but may be less preferred if alternative mRNA splicing occurs. In addition, the antisense is targeted, preferably, to sites in which mRNA secondary structure is not expected (see, e.g., Sainio et al., Cell Mol. Neurobiol. 14(5):439-457, 1994) and at which proteins are not expected to bind.

In one set of embodiments, the antisense oligonucleotides of the invention may be composed of “natural” deoxyribonucleotides, ribonucleotides, or any combination thereof. That is, the 5′ end of one native nucleotide and the 3′ end of another native nucleotide may be covalently linked, as in natural systems, via a phosphodiester internucleoside linkage. These oligonucleotides may be prepared by art recognized methods which may be carried out manually or by an automated synthesizer. They also may be produced recombinantly by vectors.

In preferred embodiments, however, the antisense oligonucleotides of the invention also may include “modified” oligonucleotides. That is, the oligonucleotides may be modified in a number of ways which do not prevent them from hybridizing to their target but which enhance their stability or targeting or which otherwise enhance their therapeutic effectiveness.

The term “modified oligonucleotide” as used herein describes an oligonucleotide in which (1) at least two of its nucleotides are covalently linked via a synthetic internucleoside linkage (i.e., a linkage other than a phosphodiester linkage between the 5′ end of one nucleotide and the 3′ end of another nucleotide) and/or (2) a chemical group not normally associated with nucleic acid molecules has been covalently attached to the oligonucleotide. Preferred synthetic internucleoside linkages are phosphorothioates, alkylphosphonates, phosphorodithioates, phosphate esters, alkylphosphonothioates, phosphoramidates, carbamates, carbonates, phosphate triesters, acetamidates, carboxymethyl esters and peptides.

The term “modified oligonucleotide” also encompasses oligonucleotides with a covalently modified base and/or sugar. For example, modified oligonucleotides include oligonucleotides having backbone sugars which are covalently attached to low molecular weight organic groups other than a hydroxyl group at the 3′ position and other than a phosphate group at the 5′ position. Thus modified oligonucleotides may include a 2′-O-alkylated ribose group. In addition, modified oligonucleotides may include sugars such as arabinose instead of ribose.

The present invention, thus, contemplates pharmaceutical preparations containing modified antisense molecules that are complementary to and hybridizable with, under physiological conditions, nucleic acid molecules encoding EDG receptor, sphingosine kinase or sphingosine-1-phosphate phosphatase polypeptides, together with pharmaceutically acceptable carriers. Antisense oligonucleotides may be administered as part of a pharmaceutical composition. In this latter embodiment, it is preferable that a slow intravenous administration be used. Such a pharmaceutical composition may include the antisense oligonucleotides in combination with any standard physiologically and/or pharmaceutically acceptable carriers which are known in the art. The compositions should be sterile and contain a therapeutically effective amount of the antisense oligonucleotides in a unit of weight or volume suitable for administration to a patient. The term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art.

The invention also embraces the use of gene therapy to increase the expression of EDG receptors (preferably EDG-3 receptor), sphingosine kinase and/or sphingosine-1-phosphate phosphatase. The procedure for performing ex vivo gene therapy is outlined in U.S. Pat. No. 5,399,346 and in exhibits submitted in the file history of that patent, all of which are publicly available documents. In general, it involves introduction in vitro of a functional copy of a gene into a cell(s) of a subject which, in some instances, contains a defective copy of the gene, and returning the genetically engineered cell(s) to the subject. The functional copy of the gene is under operable control of regulatory elements which permit expression of the gene in the genetically engineered cell(s). Numerous transfection and transduction techniques as well as appropriate expression vectors are well known to those of ordinary skill in the art, some of which are described in PCT application WO 95/00654. In vivo gene therapy using vectors such as adenovirus also is contemplated according to the invention.

The following examples are included for purposes of illustration and are not intended to limit the scope of the invention.

### EXAMPLES

### Introduction

Lipid mediators, such as sphingosine-1-phosphate (S1P) and lysophosphatidic acid, derived from membrane sphingolipids and glycerophospholipids, are released by activated platelets (Yatomi, Y., et al., J. Biochem. (Tokyo) 121: 969-973 (1997) and affect the maturation and function of cultured endothelial and smooth muscle cells. (Lee, M. J., et al., Cell 99: 301-312 (1999); English, D., et al., FASEB J. 14: 2255-2265 (2000); Liu, Y., et al., J. Clin. Invest. 106: 951-961 (2000); Igarashi, J. et al., J. Biol. Chem. 275: 32363-32370 (2000)) They have gained increasing attention since the discovery of their high affinity G-protein coupled receptors, termed Endothelium Differentiation Gene-related receptors (EDG receptors). (Hla, T. et al., J. Biol. Chem. 265: 9308-9313 (1990); Spiegel, S. et al., Biochim. Biophys. Acta 1484: 107-116 (2000)) EDG-receptors are reportedly expressed by endothelial and vascular smooth muscle cells. (Hla, T. et al., J. Biol. Chem. 265: 9308-9313 (1990); Okazaki, H., et al., Biochem. Biophys. Res. Commun. 190: 1104-1109 (1993)) However, the role of lipid mediators in regulating vascular tone has not been established nor has this system been studied under pathological conditions. Therefore in vitro and in vivo studies were performed to determine whether lipid mediators regulate vascular contractility.

### Methods and Materials

### Reagents

Sphingosine-1-phosphate, dihydrosphingosine-1-phosphate, sphingosine, dimethylsphingosine (Avanti Polar Lipids), lysophosphatidic acid (Biomol) and sphingosylphosphorylcholine (Calbiochem) were dissolved as millimolar solutions in 4 mg/ml fatty acid free bovine serum albumin. Pertussis toxin was from Sigma, C. difficile toxin B was from List Biological Laboratories. 7.5 μg (in 66 μl water) of C. botulinum C₃ exoenzyme (Biomol) were mixed with 25 μg liposome (Transfectam, Promega), resuspended in 0.5 ml physiological solution and applied directly onto two arterial preparations during 4 h, at room temperature.

### Measurement of Contractile Tension in Isolated Arteries

Arterial segments (1.5-2 mm) in physiological solution (mM: NaCl, 118; KCl, 4.6; NaHCO₃, 25; MgSO₄, 1.2; KH₂PO₄, 1.2; CaCl₂, 2.5; glucose, 10; EDTA, 0.025; pH 7.4 at 37° C., 95% O₂-5% CO₂) were threaded onto 40 μm stainless steel wires (25 μm for mouse basilar) and mounted in a isometric myograph (601M, Danish Myo Technology). Preparations were equilibrated unstretched for 30 min. The normalized passive resting force and the corresponding diameter were determined as published. (Mulvany, M. J. et al., Circ. Res. 41: 19-26 (1977)) Contractile responses were recorded with an acquisition software (Myodaq and Myodata, Danish Myo Technology). The preparations were then challenged with 100 mM KCl, 5-hydroxytryptamine (Sigma) or sphingolipids. Contractions were expressed in % of the KCl-evoked contraction. Sphingosine, dimethylsphingosine or suramin (Calbiochem) were added 30 min prior to 5-HT or S1P.

### RNA Extraction and RT-PCR

Total RNA was isolated from pools of 4 arteries with an RNeasy™ kit (Qiagen); 1.0 μg RNA samples were reverse-transcribed using random hexamer-mixed primers and the Omniscript™ kit (Qiagen). Primers for edg-1, -3, -5, -8 and spp1 were designed based on the Genbank sequences as follows: 5′-ATG GTG TCC TCC ACC AGC ATC CC-3′ (sense) (SEQ ID NO:1) and 5′-TTA AGA AGA AGA ATT GAC GTT TCC-3′ (antisense) (SEQ ID NO:2) for rat edg-1; 5′-CGG CAT AGC CTA CAA GGT CA-3′ (sense) (SEQ ID NO:3) and 5′-GAT CAC TAC GOT CCG CAG AA-3′ (antisense) (SEQ ID NO:4) for rat edg-3; 5′-ATG GGC GGT TTA TAC TCA GAG T-3′ (sense) (SEQ ID NO:5) and 5′-TCA GAC CAC TGT GTT GCC CTC-3′ (antisense) (SEQ ID NO:6) for rat edg-5; 5′-ATC TGT GCG CTC TAT GCA AG-3′ (sense) (SEQ ID NO:7) and 5′-TCT CGG TTG GTG AAG GTG TA-3′ (antisense) (SEQ ID NO:8) for rat edg-8; 5′-TGC CGC TCT ACT ACC TGT TC-3′ (sense) (SEQ ID NO:9) and 5′-AAT CTC AGC CGT GTC TCC TC-3′ (antisense) (SEQ ID NO:10) for mouse spp1. The primers for rat gapdh were 5′-TAA AGG GCA TCC TGA GCT ACA CT-3′ (sense) (SEQ ID NO:11) and 5′-TTA CTC CTT GGA GGC CAT GTA GG-3′ (antisense) (SEQ ID NO:12). Amplified DNA fragments were electrophoresed on agarose gel and visualized with ethidium bromide. PCR products were checked with restriction enzymes. The density of bands was analyzed with an image analysis system (M4, Imaging Research, Inc). Since rat spp1 has not yet been cloned, the PCR product obtained with mouse based primers was sequenced (Genbank No. AF329638) and revealed 95% identity with the mouse enzyme.

### Cloning and Viral Gene Transfer of edg-3 and edg-5 Antisense to Basilar Artery

Human edg-3 was amplified by PCR from genomic DNA; rat edg-5 was amplified by RT-PCR from total RNA isolated from rat basilar arteries. The primers were designed to add a PmeI site at the 5′- and 3′-ends: 5′-GGG GTT TAA ACA TGG CAA CTG CCC TCC C-3′ (sense) (SEQ ID NO:13) and 5′-GGG GTT TAA ACT CAG TTG CAG AAG ATC C-3′ (antisense) (SEQ ID NO:14) for edg-3 and 5′-GGG GTT TAA ACA TGG GCG GTT TAT ACT CAG-3′ (sense) (SEQ ID NO:15) and 5′-GGG GTT TAA ACT CAG ACC ACT GTG TTG CCC-3′ (antisense) (SEQ ID NO:16) for edg-5. PCR products were cloned into the PmeI site of the adenovirus shuttle vector (pQBI-AdCMV5-GFP, Quantum Biotechnologies). Plasmids were transfected into 293A cells together with linearized adenoviral genome (Quantum Biotechnologies), using the calcium phosphate method; cells and medium were harvested after plaque formation. The virus, amplified in 293A cells, was purified by double cesium chloride gradient. Basilar artery segments were incubated for 18 h in DMEM (containing 10% fetal bovine serum) with or without 10⁹ PFU/ml adenovirus (empty vector or adenovirus bearing edg-3 or edg-5 antisense), at 37° C. in 5% CO₂. The segments were placed in medium without adenovirus and incubated for 48 h. These conditions resulted in intense expression of beta-galactosidase activity in preliminary experiments using adenovirus/Lac Z. After treatment, the arterial segments were mounted into the wire myograph and tested for responses to KCl, 5-HT and S1P.

### In vivo Experiments

Pentobarbital-anaesthetized mechanically-ventilated male rats (250-300 g, Charles River) were maintained at 37.0±0.5° C. A femoral vein and artery were cannulated to monitor mean arterial blood pressure, heart rate and arterial blood gases. The left common carotid artery was ligated. The animals were placed in a stereotaxic frame and relative cerebral blood flow (rCBF) was measured by a laser Doppler flow probe (Model PF2B, Perimed) affixed to the thinned skull above the vascular territory of the left middle cerebral artery (2 mm posterior to Bregma, 3 mm lateral to midline). Changes in rCBF were expressed as a percentage of baseline and recorded for 20 minutes beginning at the onset of drug or vehicle infusion. S1P, DHS1P or vehicle were infused (100 μl/2 minutes) into the left internal carotid artery. Some rats were pretreated with suramin via infusion (200 μl/2 min) into the left femoral vein.

### Focal Embolic Cerebral Ischemia

Focal embolic cerebral ischemia was induced in isoflurane-anaethetized rats. (Zhang. R. L., et al., Brain Res. 766: 83-92 (1997)). Briefly, the middle cerebral artery was occluded by an embolus injected via an intraluminal catheter. When used, DMS was infused i.v. (5 mg/kg, 1 ml/15 min, 5 min before clot injection). Animals were sacrificed 24 h later and the brain sections stained with triphenyltetrazolium chloride for infarct volume measurement.

### Results

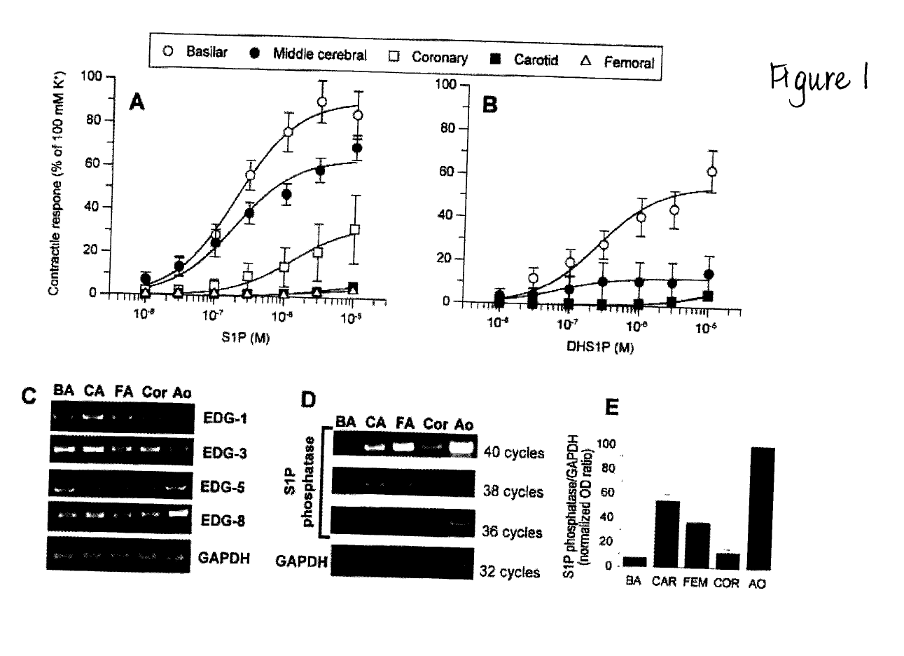

S1P was found to be a preferential constrictor of cerebral blood vessels. Sphingosine-1-phosphate evoked robust contraction of isolated rat basilar and middle cerebral arteries, with a maximum effect comparable to that of 5-hydroxytryptamine (FIG. 1a; Table 1). By contrast, coronary arteries were weakly constricted, whereas carotid and femoral arteries were unresponsive. The S1P analogue, dihydrosphingosine-1-phosphate (DHSlP) evoked a similar constrictor pattern of activity, again only in cerebral arteries, although less effectively than S1P (FIG. 1b). S1P was also able to constrict mouse and rabbit basilar arteries, but not mouse aorta (not shown).

TABLE 1
In vitro contractile parameters of arteries isolated from rat.

| | | Contractile | Contractile response | | Contractile response | | Contractile response | |
|---|---|---|---|---|---|---|---|---|
| | Normalized | response to | to 5-HT | | to S1P | | to DHS1P | |
| Artery | Diameter | KCl | EC₅₀ | Eₘₐₓ | EC₅₀ | Eₘₐₓ | EC₅₀ | Eₘₐₓ |
| (n)* | (μm) | (mN/mm) | (nM) | (% KCl) | (nM) | (% KCl) | (nM) | (% KCl) |
| Basilar | 319 ± | 1.70 ± | 63 ± | 99.5 ± | 280 ± | 96.7 ± | 240 ± | 58.8 ± |
| (20, 10, 10) | 10 | 0.22 | 17 | 5.1 | 42 | 8.4 | 47 | 9.4 |
| Middle | 199 ± | 1.05 ± | 44 ± | 73.2 ± | 350 ± | 77.6 ± | n.d. | n.d. |
| Cerebral | 7 | 0.26 | 2 | 7.3 | 80 | 9.2 | | |
| (7, 7, 0) | | | | | | | | |
| Coronary | 378 ± | 1.87 ± | 242 ± | 119.3 ± | n.d. | 16.9 ± | n.d. | 15.9 ± |
| (7, 4, 3) | 13 | 0.27 | 65 | 12.4 | | 7.0 | | 8.4 |
| Carotid | 1147 ± | 2.28 ± | 3179 ± | 101.1 ± | n.d. | 5.2 ± | n.d. | 5.9 ± |
| (9, 6, 3) | 22 | 0.30 | 1346 | 9.8 | | 3.4 | | 4.6 |
| Femoral | 552 ± | 4.52 ± | 122 ± | 133.2 ± | n.d. | 3.6 ± | n.d. | 6.3 ± |
| (7, 4, 3) | 45 | 0.83 | 33 | 3.5 | | 1.9 | | 5.0 |

EC₅₀ is the concentration of drug producing 50% of the maximum effect. Emax is the maximum contractile response, expressed in % of the contraction evoked by 100 mM KCl in the same preparation. n.d.=not determined.

The numbers in parenthesis indicate the number of preparations challenged with 5-HT, S1P or DHS1P, respectively.

S1P is an endogenous agonist of EDG-1, -3, -5 and -8 receptor subtypes. (Ancellin, N. et al., J. Biol. Chem. 274: 18997-19002 (1999); Im, D.S., et al., J. Biol. Chem. 275: 14281-14286 (2000)) RT-PCR analysis revealed transcripts for these four subtypes in arteries from multiple organs (FIG. 1c). The mRNA for each receptor subtype did not vary among arteries, ruling out differential expression as a cause for the selective S1P cerebral vasoconstriction. It was therefore investigated whether selective expression of S1P phosphatase accounts for the selectivity of S1P's action on cerebral vessels. S1P phosphatase is a membrane-bound ectoenzyme, bearing an extracellular catalytic site. (Mandala, S. M., et al., Proc. Natl. Acad. Sci. USA. 97: 7859-7864 (2000)) An increase in S1P phosphatase would reduce the availability of S1P for receptor binding (Kenakin, T. P. et al., Naunyn-Schmiedeberg's Arch. Pharmacol. 335: 103-108 (1987)) and increase the concentration of sphingosine, which behaves as an antagonist to S1P (see below). As shown in FIG. 1 (d, e), S1P phosphatase mRNA was abundant in aorta, carotid and femoral arteries, but scarce in basilar and coronary arteries. Hence, S1P phosphatase mRNA was high in arteries which were unresponsive (carotid, femoral) and low in arteries which constricted to S1P (basilar and, to a lesser extent, coronary).

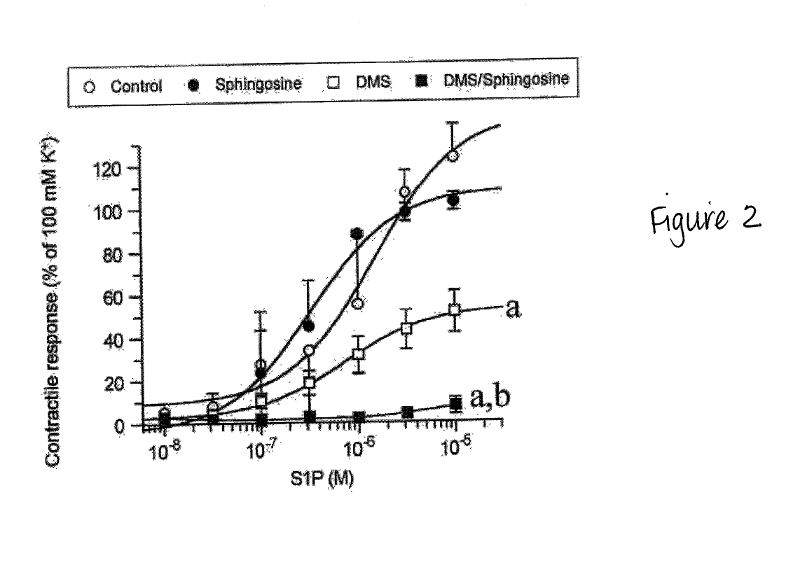

Sphingosine and sphingosine-1-phosphate are antagonists to one another and interconverted by sphingosine kinase and S1P phosphatase. The activity of sphingosine kinase and the balance between S1P and its precursor sphingosine have been reported to play a role in the regulation of apoptosis (Cuvillier, O., et al., Nature 381: 800-803 (1996); Goetzl, E. J., et al., J. Immunol. 162: 2049-2056 (1999)) and the allergic responsiveness of mast cells. (Prieschl, E. E., et al., J. Exp. Med. 190: 1-8 (1999)) It was then investigated whether sphingosine blocks the vasoconstriction of cerebral arteries induced by S1P. Basilar arteries were preincubated with or without sphingosine. When sphingosine was incubated together with sphingosine kinase inhibitor N,N-dimethyl-sphingosine (DMS) (which inhibits conversion of sphingosine to S1P (Yatomi, Y., et al., Biochemistry 35: 626-633 (1996))), the contractile response to S1P was fully blocked (FIG. 2). Hence, sphingosine completely blocks the contractile response to S1P. Therefore a balance between sphingosine and S1P can exert critical control of cerebrovascular tone.

Stimulation of EDG receptors by S1P activates Gᵢ and Gₒ heterotrimeric G proteins (for EDG-1 and EDG-8 receptors) or G_{q} and G_{12/13} proteins (for EDG-3 and EDG-5 receptors). (Ancellin, N. et al., J. Biol. Chem. 274: 18997-19002 (1999); Im, D. S., et al., J. Biol. Chem. 275: 14281-14286 (2000); Windh, R. T., et al., J. Biol. Chem. 274: 27351-27358 (1999)) G_{q} and G_{12/13} activation further leads to activation of small G-proteins of the Rho family. (Lee, M. J., et al., Cell 99: 301-312 (1999)) To characterize the transduction pathway in S1P-induced vasoconstriction, segments of basilar artery were treated, in vitro, with bacterial toxins specifically affecting G_{i/o} (B. Pertussis toxin) or Rho (C. Difficile toxin B or C. Botulinum C₃ exoenzyme). Incubation with Pertussis toxin did not modify the S1P-induced vasoconstriction, but (as expected) decreased the response to the 5-HT₁ receptor agonist 5-carboxamidotryptamine (in % of KCl-evoked contraction, from 92±10 to 30±2, n=4, p<0.05). In contrast, incubation with either toxin B or C₃ exoenzyme produced a significant decrease of the S1P-induced vasoconstriction (in % of KCl-evoked contraction, toxin B, from 81±9 to 53±6, n=8, p<0.05; C₃ exoenzyme, from 70±2 to 35±10, n=2). These results indicate that small G proteins of the Rho family are critical for S1P induced vasoconstriction, suggesting that EDG-3 and/or EDG-5 (Ancellin, N. et al., J. Biol. Chem. 274: 18997-19002 (1999); Im, D. S., et al., J. Biol. Chem. 275: 14281-14286 (2000)) mediate this response.

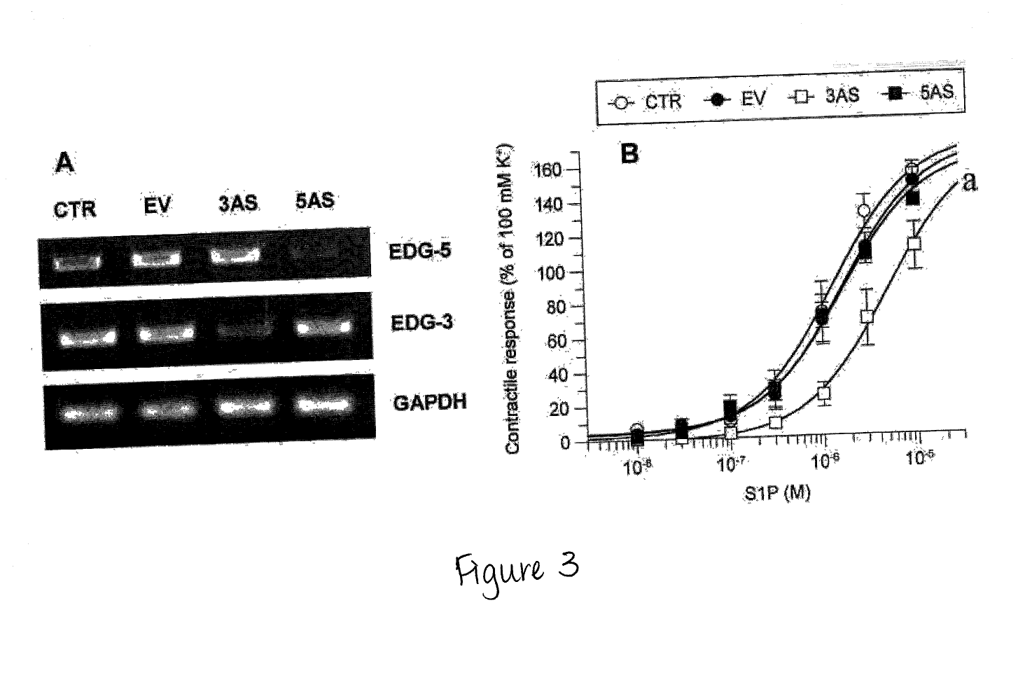

Based on these results, the relevant receptor was identified using a strategy of antisense delivery through an adenoviral vector system. Recombinant adenoviral vectors were generated bearing the antisense sequences of the open reading frames for either edg-3 or edg-5 under the control of a cytomegalovirus promoter. Basilar artery segments were treated, in vitro, with 10⁹ PFU/ml adenovirus, and further tested for the contractile responsiveness to S1P. To verify that the antisense treatment reduced edg-mRNA, edg-3 and edg-5 mRNA levels were measured by RT-PCR in adenovirus-treated basilar arteries. Gene delivery of either edg-3 or edg-5 antisense specifically reduced the respective RT-PCR product by 80% -90% (FIG. 3a). In preparations treated with edg-3 antisense the concentration-response curve to S1P was significantly shifted to the right (p<0.05 vs. empty vector; FIG. 3b). The empty vector and the adenoviruses bearing edg-3 or edg-5 antisense did not modify the contractile response to 5-HT (not shown). These results indicate that at least EDG-3 receptor mediates the vasoconstrictor response to S1P in cerebral blood vessels.

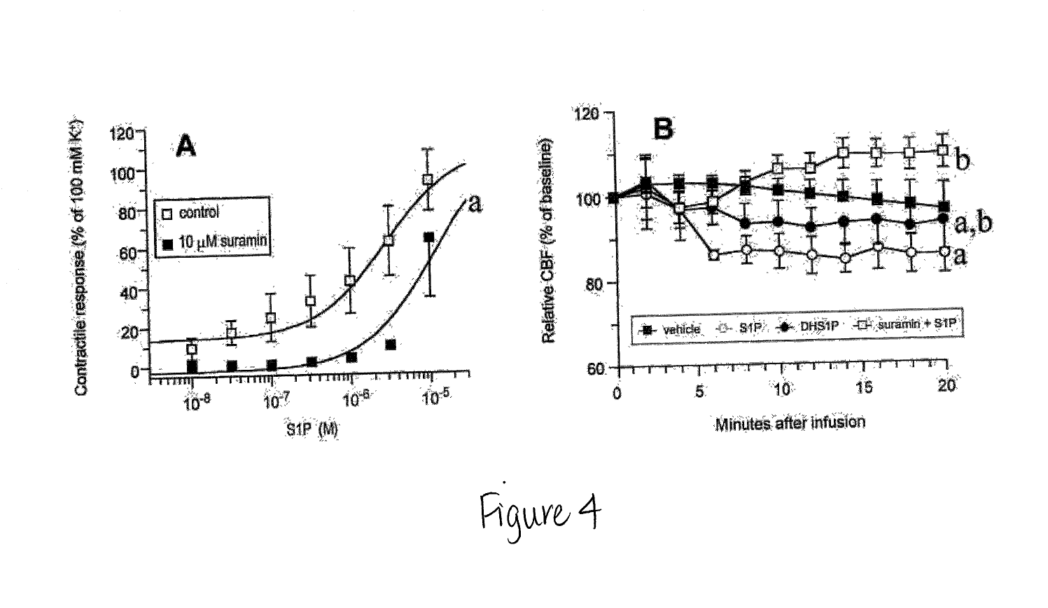

The effect of suramin on S1P-induced vasoconstriction of cerebral arteries was then investigated. Suramin is a molecule that antagonizes EDG-3 but not EDG-5 receptors (Ancellin, N. et al., J. Biol. Chem. 274: 18997-19002 (1999)). Preincubation of basilar artery with suramin markedly depressed the contractile response to S1P (FIG. 4a), whereas it did not modify the response to 5-HT (not shown). In vivo, S1P constricted cerebral blood vessels as well. Following intracarotid S1P injection to anesthetized rats, relative cerebral blood flow (rCBF) decreased in cerebral cortex as measured by laser Doppler flowmetry (FIG. 4b). DHS1P reduced rCBF, although less potently (FIG. 4b). Pretreatment with suramin abolished S1P's effect on rCBF (FIG. 4b). Suramin therefore antagonized the S1P-induced vasoconstriction, indicating the importance of the EDG-3 receptors to both in vivo and in vitro responses.

Until now, most EDG receptor characterization has been carried out in cell culture (Lee, M. J., et al., Cell 99: 301-312 (1999); Hla, T. et al., J. Biol. Chem. 265: 9308-9313 (1990); Postma, F. R., et al., EMBO J. 15: 2388-2392 (1996)) often in EDG receptor overexpressing transfectants. (Ancellin, N. et al., J. Biol. Chem. 274: 18997-19002 (1999); Kon, J., et al., J. Biol. Chem. 274: 23940-23947 (1999); Murata, N., et al., Anal. Biochem. 282: 115-120 (2000); Van Brocklyn, J. R., et al., J. Biol. Chem. 274: 4626-4632 (1999)) The present study provides evidence that S1P is a preferential vasoconstrictor in cerebral arteries. The vasoconstrictor effect in cerebral arteries occurs, in vitro, in the submicromolar range (S1P's EC₅₀ for rat basilar artery: 280 nM, Table 1) and this is of particular relevance, because S1P plasma concentrations have been reported to be 200 nM-350 nM. (Yatomi, Y., et al., J. Biochem. (Tokyo) 121:969-973(1997); Murata, N., et al., Anal. Biochem. 282: 115-120 (2000)) S1P levels are even higher in platelets (Yatomi, Y., et al., Biochemistry 35: 626-633 (1996)) therefore the local concentration of S1P could increase up to 1 μM upon platelet activation which is sufficient to evoke vasospasm. Platelet reactivity has a role in the initiation and progression of focal cerebral ischemia. (del Zoppo, G. J. Neurology 51 (Suppl. 3): S9-14 (1998)) Although vasoconstriction may be beneficial during physiological hemostasis, it would be detrimental in the context of ischemic (thrombo-embolic) injury. S1P released from platelets could therefore play a major role in diminishing cerebral blood flow in thrombo-embolic stroke. In this context, S1P receptor antagonism or sphingosine kinase inhibition could increase cerebral blood flow without lowering arterial blood pressure. Consistent with this view, it was observed that in a rat embolic clot model, pre-treatment with the sphingosine kinase inhibitor DMS significantly reduced cerebral infarct size by a third (vehicle, 392±52 mm³; DMS, 262±23 mm³; p<0.05; mean±sem; n=6 per group).

### Conclusions

The sphingolipid sphingosine-1-phosphate acts as a messenger through stimulation of G-protein coupled EDG (Endothelial Differentiation Gene) receptors. These experiments indicate that sphingosine-1-phosphate causes robust constriction of isolated cerebral, but not peripheral arteries. This selectivity could be explained by the lower expression of sphingosine-1-phosphate phosphatase in basilar artery, as assessed by RT-PCR. Remarkably, sphingosine fully prevents the vasoconstrictive response to sphingosine-1-phosphate. Therefore sphingosine, the precursor of sphingosine-1-phosphate, antagonizes the action of its phosphorylated derivative, whereas sphingosine kinase acts as a converting enzyme modulating vascular contractility. Basilar artery constriction by sphingosine-1-phosphate is specifically reduced by edg-3 antisense-treatment, showing that EDG-3 receptors mediate this response. Consistent with sphingosine-1-phosphate mediated constriction and release from platelets during clotting, a significant reduction in cerebral infarct size in an embolic stroke model was observed when rats were pre-treated with a sphingosine kinase inhibitor. Hence, sphingosine-1-phosphate/sphingosine/EDG-3 system provides a useful therapeutic target for the treatment of thrombo-embolic insults in brain. Moreover, EDG-3 receptor antagonists, sphingosine kinase inhibitors and drugs which promote S1P phosphatase activity are novel therapeutic strategies for the treatment of stroke and other related cerebrovascular diseases.

### Equivalents

Those skilled in the art will recognize, or be able to ascertain using no more than routine experimentation, many equivalents to the specific embodiments of the invention described herein. Such equivalents are intended to be encompassed by the following claims. A Sequence Listing follows the claims.

All references, patents and patent applications disclosed herein are incorporated by reference in their entirety.

We claim

1. A method for treating a subject having, or at risk of having, a disorder which can be treated by increased vasoconstriction or inhibition of vasodilation, comprising:
administering to a subject in need of such treatment an agent that up-regulates EDG receptor signaling in an amount effective to treat the disorder.

2. The method of claim 1, wherein the agent is a sphingosine kinase activator.

3. The method of claim 2, wherein the sphingosine kinase activator is selected from the group consisting of TNF-α, EGF and PDGF.

4. The method of claim 1, wherein the agent is an EDG receptor agonist.

5. The method of claim 4, wherein the EDG receptor agonist is selected from the group consisting of EDG-1 receptor agonist, EDG-3 receptor agonist, EDG-5 receptor agonist and EDG-8 receptor agonist.

6. The method of claim 4, wherein the EDG receptor agonist is an EDG-3 receptor agonist.

7. The method of claim 4, wherein the EDG receptor agonist is selected from the group consisting of sphingosine-1-phosphate, dihydro-sphingosine-1-phosphate, a sphingosine-1-phosphate analog, psychosine, sphingosylphosphorylcholine and lysophosphatidic acid.

8. The method of claim 4, wherein the EDG receptor agonist is selected from the group consisting of sphingosine-1-phosphate, dihydro-sphingosine-1-phosphate, and a sphingosine-1-phosphate analog.

9. The method of claim 1, wherein the agent is a sphingosine-1-phosphate phosphatase inhibitor.

10. The method of claim 1, wherein the disorder is one which can be treated with increased cerebral vasoconstriction or inhibition of cerebral vasodilation.

11. The method of claim 1, wherein the disorder is a migraine headache.

12. A method for decreasing arterial blood flow in a subject who would benefit from decreased arterial blood flow, comprising:
administering to a subject in need of such treatment an agent that up-regulates EDG receptor signaling in an amount effective to decrease arterial blood flow.

13. The method of claim 9, wherein the agent is a sphingosine kinase activator.

14. The method of claim 13, wherein the sphingosine kinase activator is TNF-α, EGF, or PDGF.

15. The method of claim 12, wherein the agent is an EDG receptor agonist.

16. The method of claim 15, wherein the EDG receptor agonist is selected from the group consisting of EDG-1 receptor agonist, EDG-3 receptor agonist, EDG-5 receptor agonist and EDG-8 receptor agonist.

17. The method of claim 15, wherein the EDG receptor agonist is an EDG-3 receptor agonist.

18. The method of claim 15, wherein the EDG receptor agonist is selected from the group consisting of sphingosine-1-phosphate, dihydro-sphingosine-1-phosphate, a sphingosine-1-phosphate analog, psychosine, sphingosylphosphorylcholine and lysophosphatidic acid.

19. The method of claim 15, wherein the EDG receptor agonist is selected from the group consisting of sphingosine-1-phosphate, dihydro-sphingosine-1-phosphate, and a sphingosine-1-phosphate analog.

20. The method of claim 12, wherein the in agent is a sphingosine-1-phosphate phosphatase inhibitor.

21. The method of claim 12, wherein the arterial blood flow is cerebral artery blood flow.

22. The method of claim 12, wherein the subject is having, or is at risk of having, a migraine headache.

23. A method for inducing vasoconstriction in a subject who would benefit from induced vasoconstriction, comprising:
administering to a subject in need of such treatment an agent that up-regulates EDG receptor signaling in an amount effective to induce vasoconstriction.

24. The method of claim 23, wherein the agent is a sphingosine kinase activator.

25. The method of claim 24, wherein the sphingosine kinase activator is TNF-α, EGF, or PDGF.

26. The method of claim 23, wherein the agent is an EDG receptor agonist.

27. The method of claim 26, wherein the EDG receptor agonist is selected from the group consisting of EDG-1 receptor agonist, EDG-3 receptor agonist, EDG-5 receptor agonist and EDG-8 receptor agonist.

28. The method of claim 26, wherein the EDG receptor agonist is an EDG-3 receptor agonist.

29. The method of claim 26, wherein the EDG receptor agonist is selected from the group consisting of sphingosine-1-phosphate, dihydro-sphingosine-1-phosphate, a sphingosine-1-phosphate analog, psychosine, sphingosylphosphorylcholine and lysophosphatidic acid.

30. The method of claim 26, wherein the EDG receptor agonist is selected from the group consisting of sphingosine-1-phosphate, dihydro-sphingosine-1-phosphate, and a sphingosine-1-phosphate analog.

31. The method of claim 23, wherein the in agent is a sphingosine-1-phosphate phosphatase inhibitor.

32. The method of claim 23, wherein the vasoconstriction is cerebral vasoconstriction.

33. The method of claim 23, wherein the subject is having, or is at risk of having, a migraine headache.

34. A method for treating a subject having, or at risk of having, a disorder which can be treated by increased vasodilation or inhibition of vasoconstriction, comprising:
administering to a subject in need of such treatment an agent that down-regulates EDG receptor signaling in an amount effective to treat the disorder.

35. The method of claim 34, wherein the agent is a sphingosine kinase inhibitor.

36. The method of claim 35, wherein the sphingosine kinase inhibitor is selected from the group consisting of methylsphingosine, N,N-dimethylsphingosine, trimethylsphingosine, D,L-threo-dihydrosphingosine, high density lipoprotein, and 3-fluoro-sphingosine analogues.

37. The method of claim 34, wherein the agent is an EDG receptor inhibitor.

38. The method of claim 37, wherein the EDG receptor inhibitor is selected from the group consisting of EDG-1 receptor inhibitor, EDG-3 receptor inhibitor, EDG-5 receptor inhibitor, and EDG-8 receptor inhibitor.

39. The method of claim 37, wherein the EDG receptor inhibitor is an EDG-3 receptor inhibitor.

40. The method of claim 37, wherein the EDG receptor inhibitor is sphingosine or suramin.

41. The method of claim 34, wherein the in agent is a sphingosine-1-phosphate phosphatase activator.

42. The method of claim 34, wherein the disorder is selected from the group consisting of stroke, subarachnoid hemorrhage and cerebral vasospasm.

43. A method for increasing arterial blood flow in a subject who would benefit from increased arterial blood flow, comprising:
administering to a subject in need of such treatment an agent that down-regulates EDG receptor signaling in an amount effective to increase arterial blood flow.

44. The method of claim 43, wherein the agent is a sphingosine kinase inhibitor.

45. The method of claim 44, wherein the sphingosine kinase inhibitor is selected from the group consisting of methylsphingosine, N,N-dimethylsphingo sine, trimethylsphingo sine, D,L-threo-dihydrosphingosine, high density lipoprotein, and 3-fluoro-sphingosine analogues.

46. The method of claim 43, wherein the agent is an EDG receptor inhibitor.

47. The method of claim 46, wherein the EDG receptor inhibitor is selected from the group consisting of EDG-1 receptor inhibitor, EDG-3 receptor inhibitor, EDG-5 receptor inhibitor and EDG-8 receptor inhibitor.

48. The method of claim 46, wherein the EDG receptor inhibitor is an EDG-3 receptor inhibitor.

49. The method of claim 46, wherein the EDG receptor inhibitor is sphingosine or suramin.

50. The method of claim 43, wherein the in agent is a sphingosine-1-phosphate phosphatase activator.

51. The method of claim 43, wherein the subject is having, or is at risk of having, a stroke, a subarachnoid hemorrhage or a cerebral vasospasm.

52. The method of claim 43, wherein the arterial blood flow is cerebral artery blood flow.

53. The method of claim 43, further comprising co-administering a second agent to the subject with a condition treatable by the second agent in an amount effective to treat the condition, whereby the delivery of the second agent to a tissue of the subject is enhanced as a result of the increased arterial blood flow.

54. The method of claim 53, wherein the second agent is selected from the group consisting of analeptic, analgesic, anesthetic, adrenergic agent, anti-adrenergic agent, amino acids, antagonists, antidote, anti-anxiety agent, anti-cholinergic, anti-convulsant, anti-depressant, anti-emetic, anti-epileptic, anti-hypertensive, anti-fibrinolytic, anti-hyperlipidemia, anti-migraine, anti-nauseant, anti-neoplastic (brain cancer), anti-obsessional agent, anti-obesity agent, anti-parkinsonian, anti-psychotic, appetite suppressant, blood glucose regulator, cognition adjuvant, cognition enhancer, dopaminergic agent, emetic, free oxygen radical scavenger, glucocorticoid, hypocholesterolemic, hypolipidemic, histamine H2 receptor antagonists, immunosuppressant, inhibitor, memory adjuvant, mental performance enhancer, mood regulator, mydriatic, neuromuscular blocking agent, neuroprotective, NMDA antagonist, post-stroke and post-head trauma treatment, psychotropic, sedative, sedative-hypnotic, serotonin inhibitor, tranquilizer, and treatment of cerebral ischemia, calcium channel blockers, free radical scavengers-antioxidants, GABA agonists, glutamate antagonists, AMPA antagonists, kainate antagonists, competitive and non-competitive NMDA antagonists, growth factors, opioid antagonists, phosphatidylcholine precursors, serotonin agonists, sodium- and calcium-channel blockers, and potassium channel openers.

55. The method of claim 53, wherein the second agent is TPA.

56. A method for inhibiting vasoconstriction in a subject who would benefit from inhibited vasoconstriction, comprising:
administering to a subject in need of such treatment an agent that down-regulates EDG receptor signaling in an amount effective to inhibit vasoconstriction.

57. The method of claim 56, wherein the agent is a sphingosine kinase inhibitor.

58. The method of claim 57, wherein the sphingosine kinase inhibitor is selected from the group consisting of methylsphingo sine, N,N-dimethylsphingo sine, trimethylsphingosine, D,L-threo-dihydrosphingosine, high density lipoprotein, and 3-fluoro-sphingosine analogues.

59. The method of claim 56, wherein the agent is an EDG receptor inhibitor.

60. The method of claim 59, wherein the EDG receptor inhibitor is selected from the group consisting of EDG-1 receptor inhibitor, EDG-3 receptor inhibitor, EDG-5 receptor inhibitor and EDG-8 receptor inhibitor.

61. The method of claim 59, wherein the EDG receptor inhibitor is an EDG-3 receptor inhibitor.

62. The method of claim 59, wherein the EDG receptor inhibitor is sphingosine or suramin.

63. The method of claim 56, wherein the in agent is a sphingosine-1-phosphate phosphatase activator.

64. The method of claim 56, wherein the subject is having or is at risk of having a stroke, a subarachnoid hemorrhage or a cerebral vasospasm.

65. The method of claim 56, wherein the vasoconstriction is cerebral vasoconstriction.

66. A method for identifying an agent that regulates vasoconstriction, comprising: selecting an agent that binds to sphingosine kinase, and
determining whether the agent that binds to sphingosine kinase modulates vasoconstriction,
wherein a change in vasoconstriction in the presence of the agent is indicative of an agent that regulates vasoconstriction.

67. A method for identifying an agent that regulates vasoconstriction comprising:
selecting an agent that binds to an EDG receptor, and
determining if the agent that binds to the EDG receptor modulates vasoconstriction
wherein a change in vasoconstriction in the presence of the agent is indicative of an agent that regulates vasoconstriction.

69. A method for identifying an agent that regulates vasoconstriction comprising:
selecting an agent that binds to a sphingosine-1-phosphate phosphatase, and
determining if the agent that binds to a sphingosine-1-phosphate phosphatase modulates vasoconstriction
wherein a change in vasoconstriction in the presence of the agent is indicative of an agent that regulates vasoconstriction.

70. A pharmaceutical preparation comprising
an agent that up-regulates EDG receptor signaling in an effective amount to treat a disorder which can be treated by increased vasoconstriction or inhibition of vasodilation, and
a pharmaceutically-acceptable carrier.

71. The pharmaceutical preparation of claim 70, wherein the agent is a sphingosine kinase activator.

72. The pharmaceutical preparation of claim 71, wherein the sphingosine kinase activator is TNF-α or EGF.

73. The pharmaceutical preparation of claim 70, wherein the agent is an EDG receptor agonist.

74. The pharmaceutical preparation of claim 73, wherein the EDG receptor agonist is selected from the group consisting of EDG-1 receptor agonist, EDG-3 receptor agonist, EDG-5 receptor agonist and EDG-8 receptor agonist.

75. The pharmaceutical preparation of claim 73, wherein the EDG receptor agonist is selected from the group consisting of sphingosine-1-phosphate, dihydro-sphingosine-1-phosphate, a sphingosine-1-phosphate analog, psychosine, sphingosylphosphorylcholine and lysophosphatidic acid.

76. The pharmaceutical preparation of claim 70, wherein the in agent is a sphingosine-1-phosphate phosphatase inhibitor.

77. The pharmaceutical preparation of claim 70, wherein the disorder is a migraine headache.

78. A pharmaceutical preparation comprising
an agent that down-regulates EDG receptor signaling in an effective amount to treat a disorder which can be treated by increased vasodilation or inhibition of vasoconstriction, and
a pharmaceutically-acceptable carrier.

79. The pharmaceutical preparation of claim 78, wherein the agent is a sphingosine kinase inhibitor.

80. The pharmaceutical preparation of claim 79, wherein the sphingosine kinase inhibitor is selected from the group consisting of methylsphingosine, N,N-dimethylsphingosine, trimethylsphingosine, D,L-threo-dihydrosphingosine, high density lipoprotein, and 3-fluoro-sphingosine analogues.

81. The pharmaceutical preparation of claim 78, wherein the agent is an EDG receptor inhibitor.

82. The pharmaceutical preparation of claim 81, wherein the EDG receptor inhibitor is selected from the group consisting of EDG-1 receptor inhibitor, EDG-3 receptor inhibitor, EDG-5 receptor inhibitor, and EDG-8 receptor inhibitor.

83. The pharmaceutical preparation of claim 81, wherein the EDG receptor inhibitor is an EDG-3 receptor inhibitor.

84. The pharmaceutical preparation of claim 81, wherein the EDG receptor inhibitor is sphingosine or suramin.

85. The pharmaceutical preparation of claim 78, wherein the agent is a sphingosine-1-phosphate phosphatase activator.

86. The pharmaceutical preparation of claim 78, wherein the disorder is selected from the group consisting of stroke, subarachnoid hemorrhage and a cerebral vasospasm.
